(19) United States
(12) Patent Application Publication
(10) Pub. No.: US 2018/0015125 A1
(43) Pub. Date: Jan. 18, 2018

(54) USE OF ADHERENT STROMAL CELLS FOR ENHANCING HEMATOPOIESIS IN A SUBJECT IN NEED THEREOF

(71) Applicant: PLURISTEM LTD., Haifa (IL)

(72) Inventor: Zami Aberman, Tel-Mond (IL)

(21) Appl. No.: 15/545,916

(22) PCT Filed: Mar. 21, 2016
(86) PCT No.: PCT/IB2016/051585

§ 371 Date: Jul. 24, 2017

Related U.S. Application Data
(60) Provisional application No. 62/150,864, filed on Apr. 22, 2015.
(60) Provisional application No. 62/136,638, filed on Mar. 23, 2015.

(51) Int. Cl. C12N 5/073; C12N 5/0775

(52) U.S. Cl. CPC A61K 35/35; C12N 5/0605; C12N 5/0667

(57) ABSTRACT

Disclosed herein are methods and compositions comprising adherent stromal cells.

Disclosed herein are methods and compositions comprising adherent stromal cells for treating hematological disorders.

### BACKGROUND

Previous publications (for example WO2012/127320, in the name of Pluristem Ltd) indicate that adherent stromal cells can treat compromised bone marrow following radiation or chemotherapy. However, a variety of hematological disorders are not mentioned in the previous publications.

### SUMMARY

Provided herein is data showing that adherent stromal cells can treat a variety of hematological disorders not mentioned in the previous publications.

In one embodiment, there is provided a method of treating incomplete engraftment of a hematopoietic stem cell (HSC) transplant, or a related syndrome, in a subject in need thereof, comprising the step of administering to the subject a pharmaceutical composition comprising adherent stromal cells (ASC), thereby treating incomplete engraftment. In certain embodiments, the ASC are derived from a placenta or from adipose tissue.

In other embodiments, there is provided a method of enhancing hematopoiesis in a subject having received an RIC HSC transplant, comprising the step of administering to the subject a pharmaceutical composition comprising ASC, thereby enhancing hematopoiesis in a subject having received an RIC transplant. In certain embodiments, the ASC are derived from a placenta or from adipose tissue.

Provided in other embodiments is a method of treating MDS, or a related disorder, in a subject in need thereof, comprising the step of administering to the subject a pharmaceutical composition comprising ASC, thereby treating MDS. In certain embodiments, the ASC are derived from a placenta or from adipose tissue.

Also provided herein is a method of reducing an incidence of AML in a subject with MDS, comprising the step of administering to the subject a pharmaceutical composition comprising ASC, thereby reducing an incidence of AML in a subject with MDS. In certain embodiments, the ASC are derived from a placenta or from adipose tissue.

In certain embodiments, the ASC described herein have been cultured in 2-dimensional (2D) culture, 3-dimensional (3D) culture, or a combination thereof. Non-limiting examples of 2D and 3D culture conditions are provided in the Detailed Description and in the Examples.

Reference herein to “growth” of a population of cells is intended to be synonymous with expansion of a cell population.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2A-D are plots of survival in vehicle-treated (empty squares) and ASC-treated (filled diamonds) mice. Plotted are all doses together (A) and mice that received the LD50 (B), LD70 (C), and LD90 (D) doses.

FIGS. 3A-L contain plots of the levels of IL-15 (Interleukin-15; UniProt No. P40933) (A-B), KC (keratinocyte chemoattractant/CXCL1; Uniprot No. P09341) (C-D), IL-6 (UniProt identifier P05231) (E-F), G-CSF (Granulocyte colony-stimulating factor; UniProt No. P09919) (G-H), EPO (Erythropoietin; UniProt identifier P01588) (I-J), and M-CSF (macrophage colony-stimulating factor 1; UniProt identifier P09603) (K-L), in the serum (A, C, E, G, I, K) and bone marrow (B, D, F, H, J, L). Mice were irradiated/vehicle-treated (IRR CA), irradiated/ASC-treated (IRR TA), sham irradiated/vehicle-treated (SHAM CA), or sham irradiated/ASC-treated (SHAM TA). Significant differences comparing IRR CA and IRR TA are denoted by circled asterisks, whereas significant differences comparing SHAM CA and IRR CA are denoted by regular asterisks. Single asterisks denote significant overall differences; asterisks above each time point denote differences at that time point only. n=2-6 mice per time point per group. Vertical axis: cytokine amount in pictograms/milliliter (pg/ml). Horizontal axis: days post-irradiation.

FIG. 4 contains plots of serum levels of several components, namely white blood cells in units of thousands/microliter (K/mcl) (A), neutrophils (K/mcl) (B), lymphocytes (K /mcl) (C), monocytes (D), red blood cells (E), platelets (F), and hemoglobin (G). Experimental groups and line patterns of datasets are as in the previous Figure. Units for vertical axis of A, B, C, D, and F are thousands/microliter (K/mcl); for E millions/mcl (M/mcl); and for G grams/deciliter (g/dL). Asterisks indicate statistically significant difference in IRR TA compared to IRR CA on day 23 post-irradiation; p<0.0001 to 0.0272.

FIG. 5 contains plots of BM levels of BM cellularity (A) and several types of precursors, namely CFU-GM (B), BFU-E (C), CFU-GEMM (D), and BM total HPC (E). Experimental groups and line patterns of datasets are as in the previous Figure.

FIG. 6 contains plots of blood components, namely white blood cells (A, D), granulocytes (B, E), and platelets (C, F) in mice that were lethally irradiated and then reconstituted with 4×10⁶ (A-C) or 8×10⁶ (D-F) syngeneic BM cells and treated with either placebo (dotted line) or ACS (solid line).

FIG. 7 contains plots of blood components, namely white blood cells (A, D), granulocytes (B, E), and platelets (C, F) in mice that were lethally irradiated and then reconstituted with 2×10⁶ (A-C) or 4×10⁶ (D-F) haploidentical BM cells and treated with either placebo (dotted line) or ACS (solid line).

FIG. 8 contains plots of the survival curve, expressed as the number of surviving mice (vertical axis) vs. number of weeks (horizontal axis) (A); and percentage of human HSC in the bone marrow (vertical axis) 8 weeks after irradiation (B) for mice that were non-lethally irradiated and then reconstituted with 5×10⁵ xenogeneic BM cells and treated with either placebo (dotted line) or ACS (solid line).

FIG. 9 contains plots of the migration rate of BM cells through a 5μ (micron) Transwell® insert towards CM derived from two different populations of placental ASC (ASC pop #1 and ASC pop #2), in units of migrated cells per well (A) or normalized to the negative control (NC), where the value of the negative control was arbitrarily set to 1 (B). SDF-1 indicates the positive control.

FIG. 10A is a survival curve, expressed as the percent survival (vertical axis) vs. number of days (horizontal axis) in mice exposed to various amounts of total body radiation, then treated with vehicle or ASC. Groups were as follows: 670 cGy/vehicle (n=10; hollow triangles), 720 cGy/vehicle (n=10; hollow circles), 770 cGy (n=20; 10 vehicle [large squares] and 10 ASC [small squares]), 850 cGy/ASC (n=10; [circled x's]), or 950 cGy/ASC (n=10; [hatched triangles]). FIG. 10B is a plot showing the dose reduction of ASC treatment (hatched squares) vs. vehicle (solid squares).

FIG. 11A is a perspective view of a carrier (or “3D body”), according to an exemplary embodiment. FIG. 11B is a perspective view of a carrier, according to another exemplary embodiment. FIG. 11C is a cross-sectional view of a carrier, according to an exemplary embodiment.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise adherent stromal cells (ASC). In some embodiments, the ASC are derived from placenta, while in other embodiments, the ASC are derived from adipose tissue. Alternatively or in addition, the ASC may be human ASC, or in other embodiments animal ASC.

In another embodiment is provided a method of treating incomplete engraftment of a hematopoietic stem cell (HSC) transplant in a subject in need thereof, comprising the step of administering to the subject a pharmaceutical composition comprising adherent stromal cells (ASC), thereby treating incomplete engraftment. In certain embodiments, the ASC are derived from a placenta or from adipose tissue. Incomplete engraftment of an HSC transplant refers, in some embodiments, to a failure to reach an absolute WBC count of at least 1×10⁹ cells/liter by 12 months after the transplant.

In another embodiment is provided a method of treating delayed engraftment of an HSC transplant in a subject in need thereof, comprising the step of administering to the subject a pharmaceutical composition comprising ASC, wherein the ASC are derived from a placenta or from adipose tissue, thereby treating delayed engraftment. Delayed engraftment of an HSC transplant refers, in some embodiments, to a failure to reach a normal WBC count within an expected time. As a non-limiting example, failure to reach an absolute WBC count of at least 1×10⁹ cells/liter within 20 days of receiving a BM transplant is considered delayed engraftment in certain populations (Trébéden-Negre H et al). In other embodiments, delayed engraftment may be defined as failure to reach a normal neutrophil count, or a normal platelet count, within an expected time. Non-limiting examples of these parameters are achievement of 3 consecutive days with an absolute neutrophil count of at least 0.5×10⁹ per liter; and achievement of 7 consecutive days with a platelet count of at least 20×10⁹ per liter without platelet transfusion (Horwitz M E et al). As will be appreciated by those skilled in the art, delayed engraftment can be assessed by a competent physician, in each particular circumstance.

In another embodiment is provided a method of treating failed engraftment of an HSC transplant in a subject in need thereof, comprising the step of administering to the subject a pharmaceutical composition comprising ASC, wherein the ASC are derived from a placenta or from adipose tissue, thereby treating failed engraftment. Failed engraftment of an HSC transplant refers, in some embodiments, to patients meeting at least one of the following criteria: (1) failure to achieve a leukocyte count of >100/μL by day +21 after transplantation, (2) failure to achieve a leukocyte count 2300/μL or an absolute neutrophil count (ANC) 2200/μL by day +28; or (3) failure to maintain a mean ANC 2500/μL for 7 days after having previously achieved an ANC of at least 500/μL at any time beyond day +28 (secondary neutropenia) (Weisdorf D J et al).

In another embodiment is provided a method of treating insufficient engraftment of an HSC transplant in a subject in need thereof, comprising the step of administering to the subject a pharmaceutical composition comprising ASC, wherein the ASC are derived from a placenta or from adipose tissue, thereby treating insufficient engraftment.

In another embodiment is provided a method of treating delayed hematological recovery following an HSC transplant in a subject in need thereof, comprising the step of administering to the subject a pharmaceutical composition comprising ASC, wherein the ASC are derived from a placenta or from adipose tissue, thereby treating delayed hematological recovery.

Also provided, in various embodiments, are compositions for treating or ameliorating failed, incomplete, insufficient, or delayed HSC engraftment, or delayed hematological recovery, comprising the described ASC. Provided in addition is use of the described ASC in the preparation of a medicament for treating or ameliorating failed, incomplete, insufficient, or delayed HSC engraftment, or delayed hematological recovery. Failed, incomplete, insufficient, and delayed HSC engraftment, and delayed hematological recovery, are known to those skilled in the art, and are described, for example, in Trébéden-Negre H et al, Weisdorf DJ et al, Horwitz M E et al, and the references cited therein.

In various embodiments, the ASC are administered to the subject at least 1 month, at least 2 months, at least 3 months, at least 4 months, at least 5 months, at least 6 months between 1-24 months, between 2-24 months, between 3-24 months, between 4-24 months, between 5-24 months, between 6-24 months, between 1-12 months, between 2-12 months, between 3-12 months, between 4-12 months, between 5-12 months, or between 6-12 months after the transplant.

In certain embodiments, the described intervention reduces the need for additional HSC transplants in the subject. Alternatively or in addition, the described intervention treats pancytopenia resulting from failed, incomplete, insufficient, or delayed HSC engraftment. In more specific embodiments, the pancytopenia comprises anemia, leukopenia, and thrombocytopenia.

In some embodiments, the HSC are derived from peripheral blood, or in other embodiments from bone marrow. In other embodiments, the HSC are derived from cord blood (which may be referred to in the art as a “cord blood transplant”). Alternatively or in addition, in various embodiments the HSC transplant is an autologous transplant, a syngeneic transplant, or an allogenic transplant. An allogeneic transplant may, in various embodiments, be from a histocompatible donor, a haploidentical donor, or an unrelated donor. In other embodiments, the transplant is a T-cell depleted graft. In certain embodiments, the T-cell depleted transplant is followed by donor lymphocyte infusion (DLI). In certain embodiments, the T-cell depleted transplant is administered after non-myeloablative therapy or RCI.

In still other embodiments, the ASC are administered to the subject without an additional HSC transplant. Alternatively, the ASC are administered with an additional HSC transplant. In various embodiments of the latter case, the ASC may be administered before, on the same day, or after the additional HSC transplant, in some embodiments within 30 days of one another.

In some embodiments, the ASC are administered to the subject between 6-18 months, between 6-16 months, between 8-18 months, between 8-16 months, between 10-18 months, between 10-16 months, between 12-18 months, or between 12-16 months after the HSC transplant.

In other embodiments is provided a method of enhancing hematopoiesis in a subject having received a reduced intensity conditioning (RIC) HSC transplant, comprising the step of administering to the subject a pharmaceutical composition comprising ASC, thereby enhancing hematopoiesis in a subject having received an RIC transplant. In certain embodiments, the ASC are derived from a placenta or from adipose tissue.

“Conditioning” refers to irradiation, chemotherapy, or another treatment that affects the viability and/or potency of hematopoietic cells of a subject.

RIC refers, in some embodiments, to conditioning intended to achieve incomplete myeloablation of a subject. Non-limiting examples of RIC are regimens meeting one or more of the following criteria (Bacigalupo A):

Total-body irradiation (TBI)≦200 cGy;

≦8 mg/kg total busulfan dose;

≦140 mg/m2 total melphalan dose;

≦10 mg/kg total thiotepa dose.

Those skilled in the art will appreciate that a competent physician is capable of determining the parameters for RIC in each particular circumstance.

In other embodiments is provided a method of enhancing recovery of hematopoietic function in a subject having received a RIC HSC transplant, comprising the step of administering to the subject a pharmaceutical composition comprising ASC, thereby enhancing recovery of hematopoietic function in a subject having received an RIC transplant. In certain embodiments, the ASC are derived from a placenta or from adipose tissue.

In other embodiments is provided a method of enhancing hematopoiesis in a subject having received an HSC transplant after non-myeloablative conditioning, comprising the step of administering to the subject a pharmaceutical composition comprising ASC, thereby enhancing hematopoiesis in a subject having received an HSC transplant after non-myeloablative conditioning. In certain embodiments, the ASC are derived from a placenta or from adipose tissue.

In other embodiments is provided a method of enhancing recovery of hematopoietic function in a subject having received an HSC transplant after non-myeloablative conditioning, comprising the step of administering to the subject a pharmaceutical composition comprising ASC, thereby enhancing recovery of hematopoietic function in a subject having received an HSC transplant after non-myeloablative conditioning. In certain embodiments, the ASC are derived from a placenta or from adipose tissue.

Also provided, in various embodiments, are compositions for enhancing hematopoiesis or recovery of hematopoietic function in a subject having received a transplant after RIC or non-myeloablative conditioning, comprising the described ASC. Provided in addition is use of the described ASC in the preparation of a medicament for enhancing hematopoiesis or recovery of hematopoietic function in a subject having received a transplant after RIC or non-myeloablative conditioning.

In various embodiments, the described intervention results in reduced rates of infection and/or reduced instances of abnormal bleeding, following the HSC transplant.

In some embodiments, the ASC are administered on the same day as the transplant. In other embodiments, the ASC are administered within 30 days, within 25 days, within 20 days, within 15 days, or within 10 days of the transplant; or 1-30 days, 1-25 days, 1-20 days, 1-15 days, 1-10 days, 2-30 days, 2-25 days, 2-20 days, 2-15 days, 2-10 days, 3-30 days, 3-25 days, 3-20 days, 3-15 days, 3-10 days, 1-7 days, 2-7 days, or 3-7 days after the transplant.

In some embodiments, the HSC are derived from peripheral blood, or in other embodiments from bone marrow. In other embodiments, the HSC are derived from cord blood (which may be referred to in the art as a “cord blood transplant”). Alternatively or in addition, in various embodiments the HSC transplant is an autologous transplant, a syngeneic transplant, or an allogenic transplant. An allogeneic transplant may, in various embodiments, be from a histocompatible donor, a haploidentical donor, or an unrelated donor. In other embodiments, the transplant is a T-cell depleted graft. In certain embodiments, the T-cell depleted transplant is followed by DLI. In certain embodiments, the T-cell depleted transplant is administered after non-myeloablative therapy or RCI.

Provided in other embodiments is a method of treating myelodysplastic syndrome (MDS) in a subject in need thereof, comprising the step of administering to the subject a pharmaceutical composition comprising ASC, thereby treating MDS. In certain embodiments, the ASC are derived from a placenta or from adipose tissue.

Also provided herein is a method of reducing an incidence of acute myeloid leukemia (AML) in a subject with MDS, comprising the step of administering to the subject a pharmaceutical composition comprising ASC, thereby reducing an incidence of AML in a subject with MDS. In certain embodiments, the ASC are derived from a placenta or from adipose tissue. As will be appreciated by those skilled in the art, a competent physician is capable of determining whether a subject has MDS.

Provided in still other embodiments is a method of treating oligoblastic leukemia in a subject in need thereof, comprising the step of administering to the subject a pharmaceutical composition comprising ASC, thereby treating oligoblastic leukemia. In certain embodiments, the ASC are derived from a placenta or from adipose tissue.

Also provided, in various embodiments, are compositions for treating MDS or oligoblastic leukemia, or reducing an incidence of AML following same, comprising the described ASC. Provided in addition is use of the described ASC in the preparation of a medicament for treating MDS or oligoblastic leukemia, or reducing an incidence of AML following same.

In various embodiments, the MDS or oligoblastic leukemia may be treatment-mediated or disease-mediated. Non-limiting examples of causative treatments are cancer and radiation. A non-limiting example of a causative disease is cancer.

As is known in the art and described in Paquette R L 2002, the incidence of MDS increases with age. Patients most commonly present with symptomatic anemia or incidentally noted peripheral blood abnormalities. Reticulocytopenic anemia is the most common laboratory abnormality; the red cells can be macrocytic, microcytic, or normocytic. Causes of megaloblastic anemia (vitamin B12 or folate deficiency) should be excluded when macrocytosis is present, and iron deficiency, anemia of chronic disease, or thalassemia minor should be considered in the setting of microcytosis.

Neutropenia and thrombocytopenia are variably present in MDS, but are more commonly associated with advanced disease. Thrombocytosis may occur in certain myelodysplastic syndrome subtypes, including those with an isolated 5q-cytogenetic abnormality or with increased numbers of ringed sideroblasts in the bone marrow.

The bone marrow biopsy is usually hypercellular for the patient's age. Erythroid dysplasia, including megaloblastic changes, binuclearity, or nuclear blebbing is a common feature of myelodysplastic syndrome. Ringed sideroblasts, abnormal erythroid precursors with iron-laden mitochondria ringing the nucleus, may be observed after Prussian blue staining.

Myeloid dysplasia may be characterized by increased numbers of immature forms (myeloid “left-shift”), or neutrophils with abnormal cytoplasmic granules or bilobed nuclei (pseudo-Pelger-Hut anomaly). Megakaryocytes may be abnormally small (micromegakaryocytes) or have abnormal nuclear morphology or ploidy. Increased numbers of bone marrow myeloblasts (>5% of cellular elements) are present in more advanced MDS. Cytogenetics are abnormal in 50% to 60% of de novo cases of myelodysplastic syndrome and are useful in prognostication.

A number of cytogenetic abnormalities (especially 5q-,7q- or -7, +8, or 20q-) are characteristically observed in MDS (Fenaux P et al). The N-ras oncogene can be mutated in myelodysplastic syndrome, albeit infrequently (Paquette R L 1993).

In some embodiments, the MDS comprises, or is accompanied by, refractory anemia (RA). In more specific embodiments, the RA comprises ring sideroblasts.

In other embodiments, the MDS comprises excess blasts, or in more specific embodiments, RA with excess blasts.

In yet other embodiments, the MDS comprises trilineage dysplasia, which may be RAEB-t (RA with excess blasts in transformation). In other embodiments, the MDS does not comprise transformation.

In still other embodiments, the MDS comprises, or is accompanied by, refractory cytopenia.

In other embodiments, the MDS comprises, or is accompanied by, unilineage dysplasia, or comprises refractory cytopenia with unilineage dysplasia.

In yet other embodiments, the MDS comprises, or is accompanied by, multilineage dysplasia, or comprises refractory cytopenia with multilineage dysplasia.

In still other embodiments, the MDS comprises, or is accompanied by, trilineage dysplasia, or comprises refractory cytopenia with trilineage dysplasia.

Alternatively or in addition, the subject with MDS has a marrow cytogenetic abnormality.

In still other embodiments, the MDS comprises chronic myelomonocytic leukemia (CMML).

In still other embodiments, the MDS is classified into one of the following categories, as per the WHO (World Health Organization) Classification System: RCUD (Refractory Cytopenia with Unilineage Dysplasia), which may be RA (Refractory Anemia) that is not successfully treated with iron or vitamins; RN (Refractory Neutropenia), or RT (Refractory Thrombocytopenia); RARS (Refractory anemia with ring sideroblasts); RCMD (Refractory Cytopenia with Multilineage Dysplasia); RAEB-1 (Refractory Anemia with Excess Blasts); RAEB-2 (Refractory Anemia with Excess Blasts 2); Isolated Del 5q (Deletion 5q); RCC (Refractory cytopenia in childhood); or Unclassified MDS.

As is known in the art, HSC transplants may be performed for a variety of causes, including but not limited to osteopetrosis, Fanconi anemia, breast cancer, severe combined immunodeficiency disorder (SCID), Hodgkin's' lymphoma, multiple myeloma, severe aplastic anemia, thalassemia major, or leukemias such as AML with MDS-like features (AML/MDS), MDS, CML, ALL, NHL, and AML.

In other embodiments is provided a method of treating acute myeloid leukemia (AML) in a subject in need thereof, comprising the step of administering to said subject a pharmaceutical composition comprising ASC, wherein said ASC are derived from a placenta or from adipose tissue, thereby treating AML. In alternative embodiments, CM from the ASC is administered.

In still other embodiments is provided a method of reducing an incidence of GI toxicity in a subject with AML, comprising the step of administering to said subject a pharmaceutical composition comprising ASC, wherein said ASC are derived from a placenta or from adipose tissue, thereby reducing an incidence of GI toxicity in a subject with AML. In alternative embodiments, CM from the ASC is administered.

Also provided is a composition for reducing an incidence of GI toxicity in a subject with AML, comprising the described ASC. Provided in addition is use of the described ASC in the preparation of a medicament for reducing an incidence of GI toxicity in a subject with AML.

In yet other embodiments is provided a method of reducing an incidence of infection in a subject with AML, comprising the step of administering to said subject a pharmaceutical composition comprising ASC, wherein said ASC are derived from a placenta or from adipose tissue, thereby reducing an incidence of infection in a subject with AML. In alternative embodiments, CM from the ASC is administered.

Also provided is a composition for reducing an incidence of infection in a subject with AML, comprising the described ASC. Provided in addition is use of the described ASC in the preparation of a medicament for reducing an incidence of infection in a subject with AML.

In other embodiments is provided a method of reducing a need for transfusions in a subject with AML, comprising the step of administering to said subject a pharmaceutical composition comprising ASC, wherein said ASC are derived from a placenta or from adipose tissue, thereby reducing a need for transfusions in a subject with AML. In alternative embodiments, CM from the ASC is administered.

Also provided is a composition for reducing a need for transfusions in a subject with AML, comprising the described ASC. Provided in addition is use of the described ASC in the preparation of a medicament for reducing a need for transfusions in a subject with AML.

Provided in still other embodiments is a method of treating aplastic anemia in a subject in need thereof, comprising the step of administering to the subject a pharmaceutical composition comprising ASC, thereby treating aplastic anemia. In certain embodiments, the ASC are derived from a placenta or from adipose tissue. Alternatively or additionally, the aplastic anemia may have occurred following cytotoxic cancer chemotherapy. In other embodiments, the aplastic anemia occurred or was diagnosed following a drug reaction, non-limiting examples of which are a reaction to anti-convulsant medications (e.g. carbamazepine (CBZ), valproic acid (VPA), or phenytoin), carbamazepine, hydantoins (e.g. phenytoin), phenobarbital, phenacemide, antibiotics (e.g. sulfonamide antibiotics and chloramphenicol), non-steroidal anti-inflammatory drugs (NSAID's), phenylbutazone, indomethacin, hyperthyroidism medications (e.g. methimazole and propylthiouracil), gold salts, D-penicillamine, quinacrine, acetazolamide, and arsenicals (e.g. arsphenamine). In still other embodiments, the aplastic anemia occurred or was diagnosed following exposure to chemicals, non-limiting examples of which are exposure to organic solvents (e.g. benzene, tolulene, and petroleum distillates), glue vapors, and chlorophenothane. In still other embodiments, the aplastic anemia occurred or was diagnosed following a viral infection, non-limiting examples of which are infection with Epstein-Ban virus, seronegative (non-A through non-G) hepatitis, human immunodeficiency virus (HIV), and other herpes viruses. In yet other embodiments, the aplastic anemia occurred or was diagnosed as a result of an immune disorder, non-limiting examples of which are eosinophilic fasciitis, systemic lupus erythematosus, and graft vs. host disease. In yet other embodiments, the aplastic anemia occurred or was diagnosed as a result of paroxysmal nocturnal hemoglobinuria, thymoma, pregnancy, or anorexia nervosa.

Also provided is a composition for treating aplastic anemia, comprising the described ASC. Provided in addition is use of the described ASC in the preparation of a medicament for treating aplastic anemia.

Provided in still other embodiments is a method of enhancing hematopoiesis following haplo-identical hematopoietic cell transplantation, comprising the step of administering to the subject a pharmaceutical composition comprising ASC, thereby enhancing hematopoiesis following haplo-identical hematopoietic cell transplantation. In other embodiments, there is provided a method of reducing an incident of graft rejection following haplo-identical hematopoietic cell transplantation, comprising the step of administering to the subject a pharmaceutical composition comprising ASC, thereby reducing an incident of graft rejection following haplo-identical hematopoietic cell transplantation. In still other embodiments, there is provided a method of reducing an incident of graft vs. host disease (GvHD) following haplo-identical hematopoietic cell transplantation, comprising the step of administering to the subject a pharmaceutical composition comprising ASC, thereby reducing an incident of GvHD following haplo-identical hematopoietic cell transplantation. In various embodiments, the GvHD may be mild GvHD or severe GvHD, and/or may be acute GvHD or chronic GvHD.

Also provided, in various embodiments, is a composition for enhancing hematopoiesis, reducing an incident of graft rejection, or reducing an incident of graft vs. host disease, following haplo-identical hematopoietic cell transplantation, comprising the described ASC. Provided in addition is use of the described ASC in the preparation of a medicament for enhancing hematopoiesis, reducing an incident of graft rejection, or reducing an incident of GvHD, following haplo-identical hematopoietic cell transplantation.

Provided in still other embodiments is a method of treating bone marrow failure, in a subject having received immunotherapy for cancer, comprising the step of administering to the subject a pharmaceutical composition comprising ASC, thereby treating bone marrow failure in a subject having received immunotherapy for cancer. In other embodiments, there is provided a method of treating bone marrow deficiency, in a subject having received immunotherapy for cancer, comprising the step of administering to the subject a pharmaceutical composition comprising ASC, thereby treating bone marrow deficiency, in a subject having received immunotherapy for cancer. In certain embodiments, the ASC are derived from a placenta or from adipose tissue. Alternatively or additionally, the ASC may be administered simultaneously, or during the course of, the immunotherapy, for example in order to reduce or prevent the development of bone marrow failure or bone marrow deficiency.

Provided in still other embodiments is a method of treating bone marrow failure, in a subject having received antibody therapy for cancer, comprising the step of administering to the subject a pharmaceutical composition comprising ASC, thereby treating bone marrow failure in a subject having received antibody therapy for cancer. In other embodiments, there is provided a method of treating bone marrow deficiency, in a subject having received antibody therapy for cancer, comprising the step of administering to the subject a pharmaceutical composition comprising ASC, thereby treating bone marrow deficiency, in a subject having received antibody therapy for cancer. In certain embodiments, the ASC are derived from a placenta or from adipose tissue. Alternatively or additionally, the ASC may be administered simultaneously, or during the course of, the antibody therapy, for example in order to reduce or prevent the development of bone marrow failure or bone marrow deficiency.

Also provided is a composition for treating bone marrow failure, or, in other embodiments, bone marrow deficiency, comprising the described ASC. Provided in addition is use of the described ASC in the preparation of a medicament for treating bone marrow failure, or, in other embodiments, bone marrow deficiency.

Also provided is a composition for treating aplastic anemia, comprising the described ASC. Provided in addition is use of the described ASC in the preparation of a medicament for treating aplastic anemia.

In certain embodiments, any of the described compositions further comprises a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and carrier protein are present; in certain embodiments, the osmoprotectant and carrier protein may be the same compound. Alternatively or in addition, the composition is frozen. The cells may be any embodiment of ASC mentioned herein, each of which is considered a separate embodiment.

In various embodiments, the described cells are able to exert the described therapeutic effects, each of which is considered a separate embodiment, with or without the cells themselves engrafting in the host. For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days.

Cell Sources

Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (Falcon, Becton, Dickinson, San Jose, Calif.) with washing medium. In some embodiments, the tissue treatment includes use of a DNAse, a non-limiting example of which is Benzonase from Merck.

Placental cells may be obtained, in various embodiments, from a full-term or pre-term placenta. In some embodiments, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D culturing conditions. Conditions for propagating adherent cells in 2D and 3D culture are further described hereinbelow and in the Examples section which follows.

Those skilled in the art will appreciate in light of the present disclosure that cells may be, in some embodiments, extracted from a placenta, for example using physical and/or enzymatic tissue disruption, followed by marker-based cell sorting, and then may be subjected to the culturing methods described herein.

In still other embodiments, the cells are a placental cell population that is a mixture of fetal and maternal cells and is predominantly fetal cells. In more specific embodiments, the mixture contains at least 80% fetal cells; at least 81% fetal cells; at least 82% fetal cells; at least 83% fetal cells; at least 84% fetal cells; at least 85% fetal cells; at least 86% fetal cells; at least 87% fetal cells; at least 88% fetal cells; at least 89% fetal cells; at least 90% fetal cells; at least 91% fetal cells; at least 92% fetal cells; at least 93% fetal cells; at least 94% fetal cells; at least 95% fetal cells; at least 96% fetal cells; at least 97% fetal cells; at least 98% fetal cells; at least 99% fetal cells; at least 99.1% fetal cells; at least 99.2% fetal cells; at least 99.3% fetal cells; at least 99.4% fetal cells; at least 99.5% fetal cells; at least 99.6% fetal cells; at least 99.7% fetal cells; at least 99.8% fetal cells; at least 99.9% fetal cells; at least 99.92% fetal cells; at least 99.95% fetal cells; at least 99.96% fetal cells; at least 99.97% fetal cells; at least 99.98% fetal cells; or at least 99.99% fetal cells; or contains between 90-99% fetal cells; 91-99% fetal cells; 92-99% fetal cells; 93-99% fetal cells; 94-99% fetal cells; 95-99% fetal cells; 96-99% fetal cells; 97-99% fetal cells; 98-99% fetal cells; 90-99.5% fetal cells; 91-99.5% fetal cells; 92-99.5% fetal cells; 93-99.5% fetal cells; 94-99.5% fetal cells; 95-99.5% fetal cells; 96-99.5% fetal cells; 97-99.5% fetal cells; 98-99.5% fetal cells; 90-99.9% fetal cells; 91-99.9% fetal cells; 92-99.9% fetal cells; 93-99.9% fetal cells; 94-99.9% fetal cells; 95-99.9% fetal cells; 96-99.9% fetal cells; 97-99.9% fetal cells; 98-99.9% fetal cells; 99-99.9% fetal cells; 99.2-99.9% fetal cells; 99.5-99.9% fetal cells; 99.6-99.9% fetal cells; 99.7-99.9% fetal cells; or 99.8-99.9% fetal cells.

In other embodiments, the cells are a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus), and expanding the cells, e.g. using the methods described in Example 1.

As used herein the phrase “adipose tissue” refers to a connective tissue which comprises fat cells (adipocytes). Adipose tissue-derived adherent stromal cells may be extracted, in various embodiments, by a variety of methods known to those skilled in the art, for example those described in U.S. Pat. No. 6,153,432, which is incorporated herein by reference. The adipose tissue may be derived, in other embodiments, from omental/visceral, mammary, gonadal, or other adipose tissue sites. In some embodiments, the adipose can be isolated by liposuction.

In other embodiments, ASC may be derived from adipose tissue by treating the tissue with a digestive enzyme (non-limiting examples of which are collagenase, trypsin, dispase, hyaluronidase or DNAse); and ethylenediaminetetra-acetic acid (EDTA). The cells may be, in some embodiments, subjected to physical disruption, for example using a nylon or cheesecloth mesh filter. In other embodiments, the cells are subjected to differential centrifugation directly in media or over a Ficoll or Percoll or other particulate gradient (see U.S. Pat. No. 7,078,230, which is incorporated herein by reference).

In still other embodiments, the ASC are derived from bone marrow; peripheral blood; umbilical cord blood; synovial fluid; synovial membranes; spleen; thymus; mucosa (for example nasal mucosa); limbal stroma; ligaments, for example the periodontal ligament; scalp; hair follicles, testicles; embryonic yolk sac; and amniotic fluid. In some embodiments, the ASC are human ASC, while in other embodiments, they may be animal ASC.

Alternatively or additionally, the ASC may express a marker or a collection of markers (e.g. surface marker) characteristic of MSC or mesenchymal-like stromal cells. Examples of surface markers include but are not limited to CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). Examples of markers expected to be absent from stromal cells are CD3 (UniProtKB Accession Nos. P09693 [gamma chain] P04234 [delta chain], P07766 [epsilon chain], and P20963 [zeta chain]), CD4 (UniProtKB Accession No. P01730), CD34 (UniProtKB Accession No. P28906), CD45 (UniProtKB Accession No. P08575), CD80 (UniProtKB Accession No. P33681), CD19 (UniProtKB Accession No. P15391), CD5 (UniProtKB Accession No. P06127), CD20 (UniProtKB Accession No. P11836), CD11B (UniProtKB Accession No. P11215), CD14 (UniProtKB Accession No. P08571), CD79-alpha (UniProtKB Accession No. B5QTD1), and HLA-DR (UniProtKB Accession Nos. P04233 [gamma chain], P01903 [alpha chain], and P01911 [beta chain]). All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In certain embodiments, over 90% of the ASC are positive for CD29, CD90, and CD54. “Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. In other embodiments, over 85% of the described cells are positive for CD73 and CD105; and over 65% of the described cells are positive for CD49. In yet other embodiments, less than 1% of the described cells are positive for CD14, CD19, CD31, CD34, CD39, CD45, HLA-DR, and GlyA; less than 3% of the cells are positive for CD200; less than 6% of the cells are positive for GlyA; and less than 20% of the cells are positive for SSEA4. In more specific embodiments, over 90% of the described cells are positive for CD29, CD90, and CD54; over 85% of the cells are positive for CD73 and CD105; and over 65% of the cells are positive for CD49. In still other embodiments, over 90% of the described cells are positive for CD29, CD90, and CD54; over 85% of the cells are positive for CD73 and CD105; over 65% of the cells are positive for CD49; less than 1% of the cells are positive for CD14, CD19, CD31, CD34, CD39, CD45, HLA-DR, GlyA; less than 3% of the cells are positive for CD200; less than 6% of the cells are positive for GlyA; and less than 20% of the cells are positive for SSEA4.

In other embodiments, 65% or more, more than 65%, more than 70%, more than 75%, more than 80%, more than 85%, more than 90%, more than 95%, more than 96%, more than 97%, more than 98%, more than 99%, or more than 99.5% of the cells are negative for CD56 (Neural cell adhesion molecule 1; UniProtKB Accession No. P13591). All UniProtKB entries mentioned in this paragraph were accessed on Feb. 2, 2016.

In certain embodiments, the majority of the cells in the population express at least one of CD99R, CD87, CD119, CD130, CD140a, CD321, CD338, and HLA; or in other embodiments, 2 or more, 3 or more, 4 or more, 5 or more, 6 or more, 7 or more, or all 8 of the aforementioned markers is expressed by the majority of the cells. In other embodiments, at least 60%, at least 70%, at least 80%, at least 85%, at least 90%, at least 95%, at least 96%, at least 97%, at least 98%, at least 99%, or essentially all of the cells of the population express at least one of CD99R, CD87, CD119, CD130, CD140a, CD321, CD338, and HLA-A2; or in other embodiments 2 or more, 3 or more, 4 or more, 5 or more, 6 or more, 7 or more, or all 8 of the aforementioned markers are expressed by at least 60%, at least 70%, at least 80%, at least 85%, at least 90%, at least 95%, at least 96%, at least 97%, at least 98%, at least 99%, or essentially all of the cells.

Alternatively or in addition, the majority of the cells in the population are negative for at least one of CD153, CD275, and/or CD337; or at least 2 of, or all 3 of the aforementioned markers are not expressed by the majority of the cells. In other embodiments, at least 60%, at least 70%, at least 80%, at least 85%, at least 90%, at least 95%, at least 96%, at least 97%, at least 98%, at least 99%, or essentially all of the cells are negative for at least one of CD153, CD275, and/or CD337; or at least 2 of, or all 3 of the aforementioned markers are not expressed by at least 60%, at least 70%, at least 80%, at least 85%, at least 90%, at least 95%, at least 96%, at least 97%, at least 98%, at least 99%, or essentially all of the cells.

In still other embodiments, between 30-80% of the cells in the population express at least one of CD200, SSEA-4 and HLA-A2; or at least 2 of, or all 3 of the aforementioned markers are expressed by between 30-80% of the cells.

In still other embodiments, the majority of the cells in the population express one or more of CD9, CD26, CD46, CD99, CD151, CD164, and CD340 at high levels; or in other embodiments 2 or more, 3 or more, 4 or more, 5 or more, 6 or more, or all 7 of the aforementioned markers are expressed by the majority of the cells. In each of the aforementioned cases, the occurrence of each marker is measured independently, in other words without gating for any of the other mentioned markers.

In yet other embodiments, over 50%, over 60%, over 70%, over 80%, or over 90% of the cells express CD165 (Entrez Gene ID: 23449). Alternatively or in addition, less than 50%, less than 40%, less than 30%, less than 20%, or less than 10% of the cells express one or more of CD97 antigen (Uniprot Accession No. P48960), CD55 (Uniprot Accession No. P08174), and CD146 (Uniprot Accession No. P43121). All Entrez and UniProtKB entries mentioned in this paragraph were accessed on Mar. 6, 2016.

In other embodiments, each of CD73, CD29, and CD105 is expressed by more than 90% of the ASC; and the cells do not differentiate into osteocytes, under conditions where “classical” mesenchymal stem cells would differentiate into osteocytes. The MSC used for comparison in these assays are, in some embodiments, MSC that have been harvested from bone marrow (BM) and cultured under 2D conditions. In other embodiments, the MSC used for comparison have been harvested from bone marrow (BM) and cultured under 2D conditions, followed by 3D conditions. In more particular embodiments, the mesenchymal-like ASC are maternal cells. In some embodiments, the conditions are incubation for 17 days with a solution containing 0.1 mcM dexamethasone, 0.2 mM ascorbic acid, and 10 mM glycerol-2-phosphate, in plates coated with vitronectin and collagen. In yet other embodiments, each of CD34, CD45, CD19, CD14 and HLA-DR is expressed by less than 3% of the cells; and the cells do not differentiate into osteocytes, after incubation under the aforementioned conditions. In other embodiments, each of CD73, CD29, and CD105 is expressed by more than 90% of the cells, and of CD34, CD45, CD19, CD14 and HLA-DR is expressed by less than 3% of the cells; and the cells do not differentiate into osteocytes, after incubation under the aforementioned conditions. In still other embodiments, the conditions are incubation for 26 days with a solution containing 10 mcM dexamethasone, 0.2 mM ascorbic acid, 10 mM glycerol-2-phosphate, and 10nM Vitamin D, in plates coated with vitronectin and collagen. The aforementioned solutions will typically contain cell culture medium such as DMEM+10% serum or the like, as will be appreciated by those skilled in the art.

In other embodiments, each of CD73, CD29, and CD105 is expressed by more than 90% of the ASC; and the cells do not differentiate into adipocytes, under conditions where mesenchymal stem cells would differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days. In yet other embodiments, each of CD34, CD45, CD19, CD14 and HLA-DR is expressed by less than 3% of the cells; and the cells do not differentiate into adipocytes, after incubation under the aforementioned conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days. The aforementioned solutions will typically contain cell culture medium such as DMEM+10% serum or the like, as will be appreciated by those skilled in the art.

In certain embodiments, in vitro, the described ASC stimulate endothelial cell proliferation (ECP), or in another embodiment inhibit T cell proliferation, or in another embodiment perform both activities. In other embodiments, in vivo, the cells stimulate angiogenesis, or in another embodiment exhibit immunosuppressive activity (in some embodiments, particularly for T cell responses), and or in another embodiment support hematopoietic stem cell (HSC) engraftment, or in other embodiments any 2 of the above in vivo characteristics, or in other embodiments all 3 of the above in vivo characteristics. Each combination is considered to be a separate embodiment. In certain embodiments, as provided herein, when 750 human umbilical cord endothelial cells (HUVEC) are incubated for 4 days under normoxic conditions at 37° C. on a layer of the ASC, proliferation of the HUVEC cells is at least 120%, at least 125%, at least 130%, at least 140%, at least 150%, and least 160%, or at least 180% of the level observed in the absence of ASC.

According to some embodiments, the described ASC are capable of suppressing an immune reaction in the subject. Methods of determining the immunosuppressive capability of a cell population are well known to those skilled in the art. For example, a mixed lymphocyte reaction (MLR) may be performed. In an exemplary, non-limiting MLR assay, cord blood (CB) mononuclear cells, for example human cells or cells from another species, are incubated with irradiated cord blood cells (iCB), peripheral blood-derived monocytes (PBMC; for example human PBMC or PBMC from another species), in the presence or absence of a cell population to be tested. CB cell replication, which correlates with the intensity of the immune response, can be measured by a variety of methods known in the art, for example by ³H-thymidine uptake. Reduction of the CB cell replication when co-incubated with test cells indicates an immunosuppressive capability. Alternatively, a similar assay can be performed with peripheral blood (PB)-derived MNC, in place of CB cells. Alternatively or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided herein, when 150,000 of the ASC are co-incubated for 48 hours with 50,000 allogeneic PBMC, followed by a 5-hour stimulation with 1.5 mcg of LPS, the amount of IL-10 secretion by the PBMC is at least 120%, at least 130%, at least 150%, at least 170%, at least 200%, or at least 300% of the amount observed with LPS stimulation in the absence of ASC.

In other embodiments, each of CD73, CD29, and CD105 is expressed by more than 90% of the described ASC; and the cells stimulate ECP. In yet other embodiments, each of CD34, CD19, and CD14 is expressed by less than 3% of the cells; and the cells stimulate ECP. In other embodiments, each of CD73, CD29, and CD105 is expressed by more than 90% of the cells, each of CD34, CD19, and CD14 is expressed by less than 3% of the cells; and the cells stimulate ECP.

In other embodiments, each of CD73, CD29, and CD105 is expressed by more than 90% of the described ASC; and the cells inhibit T cell proliferation. In yet other embodiments, each of CD34, CD19, and CD14 is expressed by less than 3% of the cells; and the cells inhibit T cell proliferation. In other embodiments, each of CD73, CD29, and CD105 is expressed by more than 90% of the cells, each of CD34, CD19, and CD14 is expressed by less than 3% of the cells; and the cells inhibit T cell proliferation.

In other embodiments, the described ASC exhibit a spindle shape when cultured under 2D conditions.

In still other embodiments, the population of cells is positive (on a population level) for expression of CD10 (neprilysin; UniProtKB Accession No. P08473), CD29, CD38 (ADP-ribosyl cyclase; UniProtKB Accession No. P28907), and CD40 (UniProtKB Accession No. P25942). Optionally, the majority of the cells also express CD90. Alternatively or in combination, the majority of the cells also express one or more, in other embodiments 2 or more, in other embodiments 3 or more, in other embodiments all 4 of: CD74 (HLA class II histocompatibility antigen gamma chain; UniProtKB Accession No. P04233), CD106 (Vascular cell adhesion protein 1 [VCAM]; UniProtKB Accession No. P19320), CD274 (Programmed cell death 1 ligand 1; UniProtKB Accession No. Q9NZQ7), and HLA-DR. Positivity for marker expression “on a population level” as used herein means that expression of each of the indicated markers is above the indicated threshold level for that particular marker. Alternatively or in combination, the population is at least 40% positive on a population level for one or more, in other embodiments 2 or more, in other embodiments 3 or more, in other embodiments 4 or more, in other embodiments all 5 of: CD42a (Platelet glycoprotein IX; UniProtKB Accession No. P14770), CD45Ra (an isotype of CD45 [Protein tyrosine phosphatase, receptor type, C]; UniProtKB Accession No. P08575), CD77 (Lactosylceramide 4-alpha-galactosyltransferase; UniProtKB Accession No. Q9NPC4), CD243 (Multidrug resistance protein 1; UniProtKB Accession No. P08183), and CD275 (ICOS ligand; UniProtKB Accession No. 075144). In further embodiments, at least 40% of the population is negative for expression of CD9 (UniProtKB Accession No. P21926). In certain embodiments, the population of cells is derived from placental tissue. All UniProtKB entries mentioned in this paragraph were accessed on Jan. 22, 2015. In certain embodiments, the cells express (and/or lack) one of the aforementioned combinations of markers and do not differentiate into osteocytes, under conditions where “classical” MSC would differentiate into osteocytes, as described herein. In other embodiments, the cells express (and/or lack) one of the aforementioned combinations of markers and do not differentiate into adipocytes, under conditions where MSC would differentiate into adipocytes, as described herein. In still other embodiments, the cells express (and/or lack) one of the aforementioned combinations of markers and do not differentiate into either osteocytes or adipocytes, under conditions where mesenchymal stem cells would differentiate into osteocytes or adipocytes, respectively.

In yet other embodiments, the population of cells is positive, on a population level, for expression of CD10, CD29, CD38, and HLA-DR. Optionally, the majority of the cells also express CD90. Alternatively or in combination, the majority of the cells also express one or more, in other embodiments 2 or more, in other embodiments 3 or more, in other embodiments all 4 of: CD74, CD106, CD274, and CD40. Alternatively or in combination, the population is at least 40% positive on a population level for one or more, in other embodiments 2 or more, in other embodiments 3 or more, in other embodiments 4 or more, in other embodiments all 5 of: CD42a, CD45Ra, CD77, CD243, and CD275. In further embodiments, at least 40% of the population is negative for expression of CD9. In certain embodiments, the population of cells is derived from placental tissue. In certain embodiments, the cells express (and/or lack) one of the aforementioned combinations of markers and do not differentiate into adipocytes.

In other embodiments, at least 30%, in other embodiments at least 40%, in other embodiments at least 50%, in other embodiments at least 60%, in other embodiments at least 70%, in other embodiments at least 80%, in other embodiments at least 90% of the cells are positive on an individual level for expression of CD10, CD29, CD38, and CD40. In other embodiments is provided a cell that is positive for expression of CD10, CD29, CD38, and CD40. Optionally, the cell(s) that expresses CD10, CD29, CD38, and CD40 also expresses CD90. Alternatively or in combination, the cell(s) that expresses CD10, CD29, CD38, and CD40 also expresses one or more, in other embodiments 2 or more, in other embodiments 3 or more, in other embodiments all 4 of: CD74, CD106, CD274, and HLA-DR. Alternatively or in combination, the cell(s) that expresses CD10, CD29, CD38, and CD40 also expresses for one or more, in other embodiments 2 or more, in other embodiments 3 or more, in other embodiments 4 or more, in other embodiments all 5 of: CD42a, CD45Ra, CD77, CD243, and CD275. In further embodiments, the cell(s) that expresses CD10, CD29, CD38, and CD40 also does not express expression of CD9. In certain embodiments, the cell(s) is derived from placental tissue. In certain embodiments, the cells express (and/or lack) one of the aforementioned combinations of markers and do not differentiate into osteocytes, under conditions where “classical” MSC would differentiate into osteocytes, as described herein. In other embodiments, the cells express (and/or lack) one of the aforementioned combinations of markers and do not differentiate into adipocytes, under conditions where MSC would differentiate into adipocytes, as described herein. In still other embodiments, the cells express (and/or lack) one of the aforementioned combinations of markers and do not differentiate into either osteocytes or adipocytes, under conditions where MSC would differentiate into osteocytes or adipocytes, respectively.

According to some embodiments, the ASC express CD200, while in other embodiments, the ASC lack expression of CD200. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 70%, 75%, 80%, 85%, 90%, 92%, 94%, 95%, 96%, 97%, 98%, 99%, or 99.5% of the adherent cells express CD200.

In still other embodiments, the cells may be allogeneic, or in other embodiments, the cells may be autologous. In other embodiments, the cells may be fresh or, in other embodiments, frozen (e.g., cryo-preserved).

Additional Method Characteristics

In certain embodiments, the described ASC have been subject to a 3D incubation, as described further herein. In more specific embodiments, the ASC have been incubated in a 2D adherent-cell culture apparatus, prior to the step of 3D culturing. In some embodiments, cells (which have been extracted, in some embodiments, from placenta, from adipose tissue, etc.) are then subjected to prior step of incubation in a 2D adherent-cell culture apparatus, followed by the described 3D culturing steps.

The phrase “two-dimensional culture” refers to a culture in which the cells are exposed to conditions that are compatible with cell growth and allow the cells to grow in a monolayer, which is referred to as a “two-dimensional culture apparatus”. Such apparatuses will typically have flat growth surfaces, in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface, in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3-dimensional attachment (carrier) substrate disposed therein, and a control apparatus, for controlling pH, temperature, and oxygen levels and optionally other parameters. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of adherent stromal cells under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference. A “stationary-bed bioreactor” refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during the standard perfusion rate of the bioreactor. In certain embodiments, the substrate may be lifted at unusually fast perfusion rates, for example greater than 200 rpm.

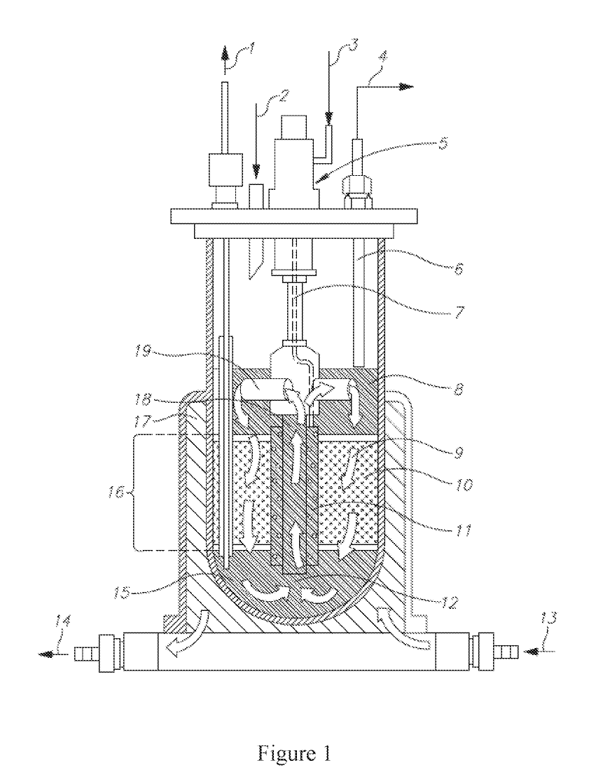

Another exemplary bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; a non-woven fibrous matrix; non-woven fibrous matrix comprising polyester; or a non-woven fibrous matrix comprising at least about 50% polyester. In more specific embodiments, the matrix may be similar to the Celligen™ Plug Flow bioreactor which is, in certain embodiments, packed with Fibra-cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the agitation speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed, washed with isotonic buffer, and processed or removed from the carriers by agitation and/or enzymatic digestion.

In certain embodiments, the bioreactor is seeded at a concentration of between 10,000-2,000,000 cells/ml of medium, in other embodiments 20,000-2,000,000 cells/ml, in other embodiments 30,000-1,500,000 cells/ml, in other embodiments 40,000-1,400,000 cells/ml, in other embodiments 50,000-1,300,000 cells/ml, in other embodiments 60,000-1,200,000 cells/ml, in other embodiments 70,000-1,100,000 cells/ml, in other embodiments 80,000-1,000,000 cells/ml, in other embodiments 80,000-900,000 cells/ml, in other embodiments 80,000-800,000 cells/ml, in other embodiments 80,000-700,000 cells/ml, in other embodiments 80,000-600,000 cells/ml, in other embodiments 80,000-500,000 cells/ml, in other embodiments 80,000-400,000 cells/ml, in other embodiments 90,000-300,000 cells/ml, in other embodiments 90,000-250,000 cells/ml, in other embodiments 90,000-200,000 cells/ml, in other embodiments 100,000-200,000 cells/ml, in other embodiments 110,000-1,900,000 cells/ml, in other embodiments 120,000-1,800,000 cells/ml, in other embodiments 130,000-1,700,000 cells/ml, in other embodiments 140,000-1,600,000 cells/ml.

In still other embodiments, between 1-20×10⁶ cells per gram (gr) of carrier (substrate) are seeded, or in other embodiments 1.5-20×10⁶ cells/gr carrier, or in other embodiments 1.5-18×10⁶ cells/gr carrier, or in other embodiments 1.8-18×10⁶ cells/gr carrier, or in other embodiments 2-18×10⁶ cells/gr carrier, or in other embodiments 3-18×10⁶ cells/gr carrier, or in other embodiments 2.5-15×10⁶ cells/gr carrier, or in other embodiments 3-15×10⁶ cells/gr carrier, or in other embodiments 3-14×10⁶ cells/gr carrier, or in other embodiments 3-12×10⁶ cells/gr carrier, or in other embodiments 3.5-12×10⁶ cells/gr carrier, or in other embodiments 3-10×10⁶ cells/gr carrier, or in other embodiments 3-9×10⁶ cells/gr carrier, or in other embodiments 4-9×10⁶ cells/gr carrier, or in other embodiments 4-8×10⁶ cells/gr carrier, or in other embodiments 4-7×10⁶ cells/gr carrier, or in other embodiments 4.5-6.5×10⁶ cells/gr carrier.

In certain embodiments, the harvest from the bioreactor is performed when at least about 10%, in other embodiments at least 12%, in other embodiments at least 14%, in other embodiments at least 16%, in other embodiments at least 18%, in other embodiments at least 20%, in other embodiments at least 22%, in other embodiments at least 24%, in other embodiments at least 26%, in other embodiments at least 28%, or in other embodiments at least 30%, of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In other embodiments, incubation of ASC may comprise microcarriers, which may, in certain embodiments, be inside a bioreactor. Microcarriers are well known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.,) Superbeads (commercially available from Flow Labs, Inc.,), and as DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

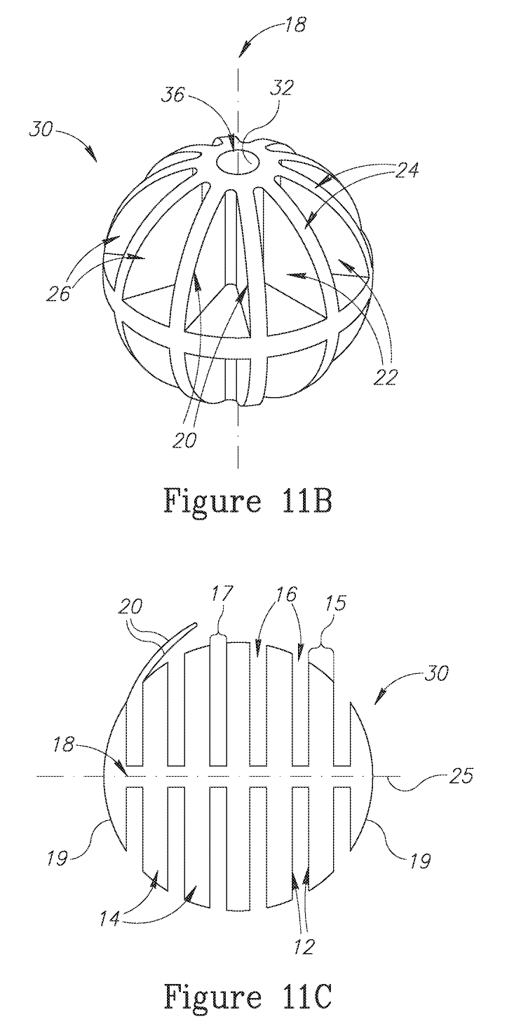

In some embodiments, with reference to FIGS. 11A-B, and as described in WO/2014/037862, published on Mar. 13, 2014, which is incorporated herein by reference in its entirety, grooved carriers 30 are used for proliferation and/or incubation of ASC. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate.

With reference to FIG. 11A, carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 11C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In other embodiments, openings 16 have a cross-sectional shape that is substantially a semicircle arc (see FIG. 11A). In still other embodiments, the central carrier axis 18 is a plane 25 that bisects the sphere, and openings 16 extend from the surface of the carrier to the proximal surface of the plane. In yet other embodiments, openings 16 extend from the surface 20 of the carrier 30 to the proximal surface of the plane and have a cross-sectional shape that is substantially a semicircle arc. In still other embodiments, carrier 30 is substantially spherical and has a largest diameter of 4-10 millimeter (mm), or between 4-9 mm, 4 5-8.5 mm, 5-8 mm, 5.5-7.5 mm, 6-7 mm, 6 1-6.9 mm, 6.2-6.8 mm, 6.3-6.7 mm, 6.4-6.6 mm, or substantially 6.5 mm In certain embodiments of the aforementioned carrier, ribs 14 are substantially flat and extend parallel to one another. In more specific embodiments, there are 3-7, 4-6, or 5 parallel ribs (not counting the extreme outer ribs 19), forming 6 openings 16 on each side of plane 25. Alternatively or in addition, the width 15 of ribs 14 and the width 17 of openings 16 are such that the ratio of rib width 15 divided by (rib width 15+opening width 17) is between 0.4-0.8, 0.45-0.75, 0.5-0.7, 0.5-0.8, 0.5-0.75, 0.55-0.65, 0.58-0.62, or substantially 0.6.

In other embodiments, carriers 30 are “3D bodies” as described in WO/2014/037862; the contents of which relating to 3D bodies are incorporated herein by reference.

As mentioned, carrier 30 may have a variety of shapes, including but not limited to spherical, cylindrical, cubical, hyperrectangular, ellipsoid, and polyhedral and/or irregular polyhedral shapes. In some embodiments, the diameter of the minimal bounding sphere (e.g. the diameter of the carrier, in the case of a spherical shape) of carrier 30 can range from 1-50 mm. In other embodiments, the outer largest dimension can range from 2-20 mm, from 3-15 mm, or from 4-10 mm. In other embodiments, the generic chord length of carriers 30 ranges from 0.5-25 mm, from 1-10 mm, from 1.5-7.5 mm, from 2-5 mm, or from 2.5-4 mm. As known to those skilled in the art, generic chord length is described inter alia in Li et al, Determination of non-spherical particle size distribution from chord length measurements. Part 1: Theoretical analysis. Chemical Engineering Science 60(12): 3251-3265, 2005)

Depending upon the overall size of carrier 30, ribs 14 and openings 16 can be variously sized. For example, ribs 14 can range in thickness from 0.1-2 mm or from 0.2 mm-1 mm. In particular, ribs 14 can be 0.4-0.6 mm, 0.5-0.7 mm, or 0.6-0.8 mm in thickness. Openings 16 can range in width from 0.01-1 mm or from 0.1-0.5 mm. In particular, openings 16 can be 0.25-0.35 mm, 0.35-0.45 mm, or 0.45-0.55 mm in width.

In preferred embodiments, the carriers provide 2D surfaces for attachment and monolayer growth over at least a majority of or all of the surface area of the multiple 2D surfaces 12, 22. Alternatively or in addition, the carriers have a surface area to volume ratio is between 3-1000 cm²/cm³, between 3-500 cm²/cm³, between 3-300 cm²/cm³, between 3-200 cm²/cm³, between 3-100 cm²/cm³, between 3-50 cm²/cm³, between 3-30 cm²/cm³, between 5-20 cm²/cm³, or between 10-15 cm²/cm³.

As shown in FIGS. 11A-B, in various embodiments, carriers 30 may be substantially spherical and have a diameter that forms the carriers' largest dimension. In some embodiments, a diameter of carrier 30 can range from 1-50 mm. In other embodiments, the diameter can range from 2-20 mm, 3-15, mm, or 4-10 mm. With reference to FIG. 11B, depending upon the overall size of carrier 30, ribs 24 and openings 26 can be variously sized. For example, ribs 24 can range in thickness from 0.1-2 mm or from 0.2-1 mm. In particular, ribs 24 can be 0.45-0.55 mm, 0 55-0.65 mm, or 0.65-0.75 mm in thickness. In some embodiments, a minimum width of openings 26 can range from 0.01-1 mm, from 0.05-0.8 mm, or from 0.1-0.5 mm. Specifically, the minimum width of openings 26 can be 0.25-0.35 mm, 0.3.5-0.45 mm, or 0.45-0.55 mm. In other embodiments, the largest cross-sectional dimension of opening 26 can range from 0.1-5 mm, from 0.2-3 mm, or from 0.5-2 mm. More particularly, opening 26 can have a largest cross-sectional dimension of 0.7.5-0.85 mm, 0.95-1.05 mm, or 1.15-0.25 mm. Further, carrier 30 includes an opening 36 extending through the carrier's center and forming additional surfaces 32, which can support monolayer growth of eukaryotic cells.

In the embodiment shown in FIG. 11A, ribs 14 are substantially flat and extend parallel to one another. In other embodiments, the ribs are in other configurations. For example, FIG. 11B illustrates carrier 30 having multiple two-dimensional surfaces 22 formed by ribs 24 in a different configuration. In particular, ribs 24 are shaped to form openings 26 that are spaced around the circumference of carrier 30, whereby openings 26 can be generally wedge shaped. Ribs 24 can extend generally radially from a central carrier axis 18 of carrier 30 to a peripheral surface of carrier 30. Carrier 30 can also include one or more lateral planes extending from the central carrier axis 18 of carrier 30 and extending generally perpendicular to ribs 24, as depicted in FIG. 11C, which is a cross-sectional view of certain embodiments of the carrier 30 of FIG. 11A.

In still other embodiments, the material forming the multiple 2D surfaces comprises at least one polymer. In more specific embodiments, the polymer is selected from a polyamide, a polycarbonate, a polysulfone, a polyester, a polyacetal, and polyvinyl chloride.

The material used to produce the described carriers can include, in various embodiments, metals (e.g. titanium), metal oxides (e.g., titanium oxide films), glass, borosilicate, carbon fibers, ceramics, biodegradable materials (e.g. collagen, gelatin, PEG, hydrogels), and or polymers. Suitable polymers may include polyamides, such as GRILAMID® TR 55 (EMS-Grivory, Sumter, SC); polycarbonates such as LEXAN® (Sabic, Pittsfield, Mass.) and Macrolon® (Bayer); polysulfones such as RADEL® PPSU (Solvay) and UDEL® PSU (Solvay); polyesters such as TRITAN® (Polyone) and PBT® HX312C; polyacetals such as CELON® (Ticana), and polyvinyl chloride. In certain embodiments, the described carriers are composed of a non-porous material, or, if pores are present, they are no larger than 20 microns, in other embodiments 10 microns, in other embodiments 5 microns, in other embodiments 3 microns, in other embodiments 2 microns, or in other embodiments 1 micron.

In more specific embodiments, cell-culture carriers are formed of injection-molded surface treatment of LEXAN® or GRILAMID®, with a smooth surface texture, using growth medium proteins and/or polylysine on LEXAN® or GRILAMID® carriers; cell-culture carriers formed of injection-molded GRILAMID® with a rough surface that was preincubated with growth medium proteins. In other embodiments, untreated LEXAN® or GRILAMID® surfaces are utilized.

In other embodiments, at least part of the carriers may be formed using a polystyrene polymer. The polystyrene may be further modified using corona discharge, gas-plasma (roller bottles and culture tubes), or other similar processes. These processes can generate highly energetic oxygen ions which graft onto the surface polystyrene chains so that the surface becomes hydrophilic and negatively charged when medium is added. Furthermore, any of the carriers may be produced at least in part from combinations of materials. Materials of the carriers can be further coated or treated to support cell attachment. Such coating and/or pretreatment may include use of collagen I, collagen IV, gelatin, poly-d-lysine, fibronectin, laminin, amine, and carboxyl.

In various embodiments, the described carriers are coated with one or more coatings. Suitable coatings may, in some embodiments, be selected to control cell attachment or parameters of cell biology. Suitable coatings may include, for example, peptides, proteins, carbohydrates, nucleic acid, lipids, polysaccharides, glycosaminoglycans, proteoglycans, hormones, extracellular matrix molecules, cell adhesion molecules, natural polymers, enzymes, antibodies, antigens, polynucleotides, growth factors, synthetic polymers, polylysine, drugs and/or other molecules or combinations or fragments of these.

Furthermore, in various embodiments, the surfaces of the carriers described herein may be treated or otherwise altered to control cell attachment and or other biologic properties. Options for treating the surfaces include chemical treatment, plasma treatment, and/or corona treatment. Further, in various embodiments, the materials may be treated to introduce functional groups into or onto the material, including groups containing hydrocarbons, oxygen, and/or nitrogen. In addition, in various embodiments, the material may be produced or altered to have a texture to facilitate settling of cells or control other cell properties. For example, in some embodiments, the materials used to produce the cell-culture carriers have a roughness on a nanometer or micrometer scale that facilitates settling of cells and/or controls other cell properties.

In certain embodiments, further steps of purification or enrichment for ASC may be performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In certain embodiments, the described method further comprises the subsequent step (following the described 3D incubation) of harvesting the ASC by removing the ASC from the 3D culture apparatus. In more specific embodiments, the harvesting process comprises agitation. In certain embodiments, the agitation is vibration, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase, Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. In more specific embodiments, the total duration of agitation during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to agitation at 0.7-6 Hertz, or in other embodiments 1-3 Hertz, during the wash step before the protease and calcium chelator are added.

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, whether or not inflammatory cytokines are added, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

Incubation with Pro-Inflammatory Cytokines

In certain embodiments, the ASC used in the described methods and compositions have been incubated with pro-inflammatory cytokines. Reference herein to one or more “pro-inflammatory” cytokines, or “inflammatory cytokines”, which is used interchangeably, implies the presence of at least one cytokine that mediates an inflammatory response in a mammalian host, for example a human host. A non-limiting list of cytokines are Interferon-gamma (IFN-gamma or IFN-gamma; UniProt identifier P01579), IL-22 (UniProt identifier Q9GZX6), Tumor Necrosis Factor-alpha (TNF-alpha; UniProt identifier P01375), IFN-alpha, IFN-beta (UniProt identifier P01574), IL-1alpha (UniProt identifier P01583), IL-1beta (UniProt identifier P01584), IL-17 (UniProt identifier Q5QEX9), IL-23 (UniProt identifier Q9NPF7), IL-17A (UniProt identifier Q16552), IL-17F (UniProt identifier Q96PD4), IL-21 (UniProt identifier Q9HBE4), IL-13 (UniProt identifier P35225), IL-5 (UniProt identifier P05113), IL-4 (UniProt identifier P05112), IL-33 (UniProt identifier 095760), IL-1RL1 (UniProt identifier Q01638), TNF-Beta (UniProt identifier P01374), IL-11 (UniProt identifier P20809), IL-9 (UniProt identifier P15248), IL-2 (UniProt identifier P60568), IL-21 (UniProt identifier Q9HBE4), Tumor Necrosis Factor-Like Ligand (TL1A; a.k.a. TNF ligand superfamily member 15; UniProt identifier O95150), IL-12 (UniProt identifiers P29459 and P29460 for the alpha- and beta subunits, respectively), and IL-18 (UniProt identifier Q14116). Additional cytokines include (but are not limited to): Leukemia inhibitory factor (LIF; UniProt identifier P15018), oncostatin M (OSM; UniProt identifier P13725), ciliary neurotrophic factor (CNTF (UniProt identifier P26441), and IL-8 (UniProt identifier P10145). All Swissprot and UniProt entries were accessed on Jul. 24, 2014, except where indicated otherwise.

Except where indicated otherwise, reference to a cytokine or other protein is intended to include all isoforms of the protein. For example, IFN-alpha includes all the subtypes and isoforms thereof, such as but not limited to IFN-alpha 17, IFN-alpha 4, IFN-alpha 7, IFN-alpha 8, and IFN-alpha 110. Some representative UniProt identifiers for IFN-alpha are P01571, P05014, P01567, P32881, and P01566. Those skilled in the art will appreciate that, even in the case of human cells, the aforementioned cytokines need not be human cytokines, since many non-human (e.g. animal) cytokines are active on human cells. Similarly, the use of modified cytokines that have similar activity to the native forms falls within the scope of the described embodiments.

In certain embodiments, the cytokine present in the described medium, or in other embodiments at least one of the cytokines present, if more than one is present, is an inflammatory cytokine that affects innate immune responses. In further embodiments, the cytokine is one of, or in other embodiments more than one, of TNF-α, IL-1alpha, IL-10, IL-12, IFN-α IFN-β, or IFN-γ.

In other embodiments, the cytokine, or in other embodiments at least one of the cytokines, if more than one is present, is an inflammatory cytokine that affects adaptive immune responses. In further embodiments, the cytokine is one of, or in other embodiments more than one, of IL-2, IL-4, IL-5, TGF-β, IL-10 or IFN-γ.

In still other embodiments, the cytokine, or in other embodiments at least one of the cytokines, if more than one is present, is a Th1 cytokine. In further embodiments, the cytokine is one of, or in other embodiments more than one, of IFN-gamma, IL-22, TNF-alpha, IL-1alpha, or IL-1beta.

In still other embodiments, the cytokine, or in other embodiments at least one of the cytokines, if more than one is present, is a Th17 cytokine. In further embodiments, the cytokine is one of, or in other embodiments more than one, of IL-17, IL-23, IL-17A, IL-17F, IL-21, IL-22, TNF-alpha, or granulocyte macrophage colony stimulating factor (GM-CSF; UniProt identifier P04141).

In yet other embodiments, the cytokine, or in other embodiments at least one of the cytokines, if more than one is present, is selected from a Th1 cytokine and a Th17 cytokine.

In still other embodiments, the cytokine, or in other embodiments at least one of the cytokines, if more than one is present, is a Th2 cytokine. In further embodiments, the cytokine is one of, or in other embodiments more than one, of IL-13, IL-5, IL-4, IL-33, IL-1RL1, TNF-alpha, and TNF-beta. In other embodiments, the cytokine is one of, or in other embodiments more than one, of IL-13, IL-5, IL-33, IL-1RL1, TNF-alpha, or TNF-beta.

In yet other embodiments, the cytokine(s) is one of, or in other embodiments more than one, of IL-11, Leukemia inhibitory factor (LIF), oncostatin M (OSM), ciliary neurotrophic factor (CNTF), granulocyte macrophage colony stimulating factor (GM-CSF), and IL-8. In further embodiments, the cytokine(s) is one or more of IL-11, LIF, OSM, CNTF, GM-CSF, or IL-8; or is one or more of IL-11, LIF, OSM, CNTF, GM-CSF, IL-8, IL-9, IL-2, IL-21.

In other embodiments, the cytokine(s) is one of, or in other embodiments more than one, of: TNF-α, IL-1beta, or TL1A.

In yet other embodiments, the cytokine(s) is one of, or in other embodiments more than one, of IL-12, IL-18, TNF-α.

In more specific embodiments, one of the aforementioned cytokines is present in the medium in an amount of 0.1-10 ng/ml; 0.15-10 ng/ml; 0.2-10 ng/ml; 0.3-10 ng/ml; 0.4-10 ng/ml; 0.5-10 ng/ml; 0.7-10 ng/ml; 1-10 ng/ml; 1.5-10 ng/ml; 2-10 ng/ml; 3-10 ng/ml; 4-10 ng/ml; 5-10 ng/ml; 0.1-5 ng/ml; 0.2-5 ng/ml; 0.3-5 ng/ml; 0.4-5 ng/ml; 0.5-5 ng/ml; 0.7-5 ng/ml; 1-5 ng/ml; 2-5 ng/ml; 0.1-3 ng/ml; 0.2-3 ng/ml; 0.3-3 ng/ml; 0.4-3 ng/ml; 0.5-3 ng/ml; 0.6-3 ng/ml; 0.8-3 ng/ml; 1-3 ng/ml; 1.5-3 ng/ml; 0.1-2 ng/ml; 0.2-2 ng/ml; 0.3-2 ng/ml; 0.4-2 ng/ml; 0.5-2 ng/ml; 0.6-2 ng/ml; 0.8-2 ng/ml; 1-2 ng/ml; 0.5-1.5 ng/ml; 0.6-1.5 ng/ml; 0.6-1.4 ng/ml; 0.7-1.3 ng/ml; 0.8-1.2 ng/ml; 0.1-0.8 ng/ml; 0.1-0.6 ng/ml; 0.1-0.5 ng/ml; 0.1-0.4 ng/ml; 0.2-1 ng/ml; 0.2-0.8 ng/ml; 0.2-0.6 ng/ml; 0.2-0.5 ng/ml; 0.2-0.4 ng/ml; 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; 2-4 ng/ml; 10-1000 ng/ml; 20-1000 ng/ml; 30-1000 ng/ml; 40-1000 ng/ml; 50-1000 ng/ml; 70-1000 ng/ml; 100-1000 ng/ml; 150-1000 ng/ml; 200-1000 ng/ml; 300-1000 ng/ml; 400-1000 ng/ml; 500-1000 ng/ml; 10-500 ng/ml; 20-500 ng/ml; 30-500 ng/ml; 40-500 ng/ml; 50-500 ng/ml; 70-500 ng/ml; 100-500 ng/ml; 200-500 ng/ml; 10-300 ng/ml; 20-300 ng/ml; 30-300 ng/ml; 40-300 ng/ml; 50-300 ng/ml; 60-300 ng/ml; 80-300 ng/ml; 100-300 ng/ml; 150-300 ng/ml; 10-200 ng/ml; 20-200 ng/ml; 30-200 ng/ml; 40-200 ng/ml; 50-200 ng/ml; 60-200 ng/ml; 80-200 ng/ml; 100-200 ng/ml; 50-150 ng/ml; 60-15 ng/ml; 60-14 ng/ml; 70-130 ng/ml; 80-120 ng/ml; 10-100 ng/ml; 10-80 ng/ml; 10-60 ng/ml; 10-50 ng/ml; 10-40 ng/ml; 20-100 ng/ml; 20-80 ng/ml; 20-60 ng/ml; 20-50 ng/ml; or 20-40 ng/ml. In still other embodiments, when more than one cytokine is present, each of them is present in an amount independently selected from the above amounts, which may be freely combined. In various other embodiments, the amounts of each of the proinflammatory cytokines present are each within one of the above ranges.

In certain embodiments, one or more of the cytokines is TNF-alpha. In more specific embodiments, the TNF-alpha may be the only cytokine present, or, in other embodiments, may be present together with 1, 2, 3, 4, 5, 6, 1-2, 1-3, 1-4, 1-5, or 1-6, or more than 6 added inflammatory cytokines, which may be, in certain embodiments, one of the aforementioned cytokines. In more specific embodiments, TNF-alpha is present in an amount of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

In some embodiments, TNF-alpha is present together with IFN-gamma. These two cytokines may be the only 2 added cytokines, or, in other embodiments, present with additional proinflammatory cytokines. In still other embodiments, IFN-gamma and TNF-alpha are each present in an amount independently selected from one of the aforementioned amounts or ranges. Each combination may be considered as a separate embodiment. In still other embodiments, the amounts of IFN-gamma and TNF-alpha are both within the range of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

As mentioned, in some embodiments, TNF-alpha is present together with one, or in other embodiments 2, 3, 4, 5, or more than 5, of the aforementioned cytokines. In still other embodiments, TNF-alpha and one, or in other embodiments more than one, of the additional cytokines is each present in an amount independently selected from one of the aforementioned amounts or ranges. Each combination may be considered as a separate embodiment. In still other embodiments, the amounts of TNF-alpha and the other cytokine(s) are both within the range of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

In certain embodiments, one or more of the cytokines is IFN-gamma. In more specific embodiments, the IFN-gamma may be the only cytokine present, or, in other embodiments, may be present together with 1, 2, 3, 4, 5, 6, 1-2, 1-3, 1-4, 1-5, or 1-6, or more than 6 added cytokines. In more specific embodiments, IFN-gamma is present in an amount of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

As mentioned, in some embodiments, IFN-gamma is present together with one of the aforementioned cytokines. These two cytokines may be the only 2 added cytokines, or, in other embodiments, present with additional proinflammatory cytokines. In still other embodiments, IFN-gamma and one, or in other embodiments more than one, of the additional cytokines is each present in an amount independently selected from one of the aforementioned amounts or ranges. Each combination may be considered as a separate embodiment. In still other embodiments, the amounts of IFN-gamma and the other cytokine(s) are both within the range of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

In certain embodiments, after the cells have been sufficiently perfused to reach the target cell concentration, perfusion is continued with cytokine-containing medium, but the rate of perfusion is adjusted to maintain homeostasis of one or more other parameters, for example glucose concentration, pH, dissolved oxygen concentration, or the like.

The various media described herein, i.e. the 2D growth medium, if applicable, the first 3D growth medium, and the second (cytokine-containing) 3D growth medium, may be independently selected from each of the described embodiments relating to medium composition. In certain embodiments, the only difference between the first and second 3D growth media is the presence of the added cytokines. In other embodiments, the first and second 3D growth media differ in other respects. In various embodiments, any medium suitable for growth of cells in a bioreactor may be used.

Those skilled in the art will appreciate that animal sera and other sources of growth factors are often included in growth media. In some cases, animal sera may contain inflammatory cytokines, which, in general, will not generally be present in large amounts. Some preparations utilize sera that are treated, for example with charcoal, so as to remove most or all of the cytokines present. In any event, reference herein to “added cytokines”, “medium containing cytokines”, or the like, does not encompass the presence of cytokines present in animal sera that is customarily included in the medium.

In certain embodiments, the ASC, prior to their ex vivo exposure to cytokines, are placenta-derived, adipose-derived, or bone marrow-derived ASC. Alternatively or in addition, the ASC are mesenchymal-like adherent stromal cells, which exhibit a marker pattern similar to “classical” MSC, but do not differentiate into osteocytes, under conditions where “classical” MSC would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where MSC would differentiate into osteocytes or adipocytes, respectively. The MSC used for comparison in these assays are, in one embodiment, MSC that have been harvested from bone marrow (BM) and cultured under 2D conditions. In other embodiments, the MSC used for comparison have been harvested from bone marrow (BM) and cultured under 2D conditions, followed by 3D conditions. In more particular embodiments, the mesenchymal-like adherent stromal cells are maternal cells, or in other embodiments are fetal cells, or in other embodiments are a mixture of fetal cells and maternal cells.

In yet other embodiments, extracellular vesicles, e.g. exosomes, secreted by the described ASC are used in the described methods and compositions. Methods of isolating exosomes are well known in the art, and include, for example, immuno-magnetic isolation, for example as described in Clayton A et al, 2001; Mathias RA et al, 2009; and Crescitelli R et al, 2013.

In some embodiments, the exosomes or other extracellular vesicles are harvested from a 3D bioreactor in which the ASC have been incubated. Alternatively or in addition, the cells are cryopreserved, and then are thawed, after which the exosomes are isolated. In some embodiments, after thawing, the cells are cultured in 2D culture, from which the exosomes are harvested. In certain embodiments, the 2D culture is performed in the presence of inflammatory cytokines, which may be, in various embodiments, any of the cytokines mentioned herein.

Pharmaceutical Compositions

Provided in addition are pharmaceutical compositions, comprising the described ASC. In other embodiments are provided pharmaceutical compositions, comprising the described exosomes.

Also provided are pharmaceutical compositions, comprising the described conditioned media. Those skilled in the art will appreciate that, in certain embodiments, various bioreactors may be used to prepare conditioned medium, including but not limited to plug-flow bioreactors, and stationary-bed bioreactors (Kompier R et al. Use of a stationary bed reactor and serum-free medium for the production of recombinant proteins in insect cells. Enzyme Microb Technol. 1991.13(10):822-7.)

The described ASC, or CM derived thereform, can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprises ASC or CM in combination with an excipient, e.g., a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of ASC mentioned herein, each of which is considered a separate embodiment.

Since non-autologous cells may in some cases induce an immune reaction when administered to a subject, several approaches may be utilized according to the methods provided herein to reduce the likelihood of rejection of non-autologous cells. In some embodiments, these approaches include either suppressing the recipient immune system or encapsulating the non-autologous cells in immune-isolating, semipermeable membranes before transplantation. In some embodiments, this may be done, in various embodiments, whether or not the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

Examples of immunosuppressive agents that may be used in the methods and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner (as detailed hereinabove). Alternatively, one may administer the pharmaceutical composition locally, for example, via injection of the pharmaceutical composition directly into an affected tissue region of a patient. In other embodiments, the cells are administered intravenously (IV), subcutaneously (SC), or intraperitoneally (IP), each of which is considered a separate embodiment. In other embodiments, the ASC or composition is administered intramuscularly; while in other embodiments, the ASC or composition is administered systemically. In this regard, “intramuscular” administration refers to administration into the muscle tissue of a subject; “subcutaneous” administration refers to administration just below the skin; and “intravenous” administration refers to administration into a vein of a subject; and “intraperitoneal” administration refers to administration into the peritoneum of a subject.

In still other embodiments, the pharmaceutical composition is administered intralymphatically, for example as described in U.S. Pat. No. 8,679,834 in the name of Eleuterio Lombardo and Dirk Buscher, which is hereby incorporated by reference.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human The dosage may vary depending upon the dosage form employed and the route of administration utilized. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The described ASC are, in some embodiments, suitably formulated as pharmaceutical compositions which can be suitably packaged as an article of manufacture. Such an article of manufacture comprises a packaging material which comprises a label describing a use in treating a disease or disorder or therapeutic indication that is mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

A typical dosage of the described ASC used alone might range, in some embodiments, from about 10 million to about 500 million cells per administration. For example, the dosage can be, in some embodiments, 10, 20, 30, 40, 50, 60, 70, 80, 90, 100, 125, 150, 175, 200, 225, 250, 275, 300, 325, 350, 375, 400, 425, 450, 475, or 500 million cells or any amount in between these numbers. It is further understood that a range of adherent stromal cells can be used including from about 10 to about 500 million cells, from about 100 to about 400 million cells, from about 150 to about 300 million cells. Accordingly, disclosed herein are therapeutic methods, the method comprising administering to a subject a therapeutically or prophylactically effective amount of ASC, wherein the dosage administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or, in other embodiments, between 150 million to 300 million cells. ASC, compositions comprising ASC, and/or medicaments manufactured using ASC can be administered, in various embodiments, in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections, or more.

It is clarified that each embodiment of the described ASC may be freely combined with each embodiment relating to a therapeutic method or pharmaceutical composition.

Furthermore, each embodiment of the described exosomes may be freely combined with each embodiment relating to a therapeutic method or pharmaceutical composition.

In still other embodiments, the described conditioned medium is used in any of the described therapeutic methods. Each embodiment of conditioned medium may be freely combined with each embodiment relating to a therapeutic method or pharmaceutical composition.

Subjects

In certain embodiments, the subject treated by the described methods and compositions is a human In other embodiments, the subject may be an animal. In some embodiments, treated animals include domesticated animals and laboratory animals, e.g., non-mammals and mammals, for example non-human primates, rodents, pigs, dogs, and cats. In certain embodiments, the subject may be administered with additional therapeutic agents or cells.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including adherent stromal cells. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1

### Culturing and Production of Adherent Placental Cells

Overview: The manufacturing process for the final cell product consisted of 2 stages: Stage 1, the intermediate cell stock (ICS) production, contains the following steps:

1. Extraction of ASCs from the placenta.
2. 2-dimensional cell growth for up to 12 population doublings.
3. Cell concentration, formulation, filling and cryopreservation. Stage 2, the thawing of the ICS and further culture, contains the following steps:
1. 2-dimensional cell growth of the thawed ICS for up to 8 additional doublings.
2. 3-dimensional cell growth in bioreactor/s and harvest from bioreactor/s up to 10 additional doublings.
3. Downstream processing: cell concentration, washing, formulation, filling and cryopreservation.

The procedure included periodic testing of the growth medium for sterility and contamination.

### Production of ICS

### Step 1-1—Extraction of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled until the initiation of the extraction process.

Within 4 hours of the delivery, the placenta was placed with the maternal side facing upwards and was cut into pieces (sized ˜1 cm³), which were washed thoroughly with isotonic buffer) containing gentamicin.
The washed pieces were incubated for 3 hours with collagenase and DNAse in isotonic buffer.
   Culture medium (DMEM], 10% filtered FBS and L-Glutamine) supplemented with gentamicin, was added, and the digested tissue was coarsely filtered through a sterile stainless steel sieve and centrifuged.
   The cells were suspended in culture medium, seeded in flasks, and incubated at 37° C. in a tissue culture incubator under humidified conditions supplemented with 5% CO₂.
   After 2-3 days, cells were washed twice with Phosphate-Buffered Saline (PBS), and the culture medium was replaced.
   Cells were incubated for an additional 4-5 days prior to the first passage.

### Step 1-2—Initial 2-Dimensional Culturing

Passage 1: Cells were detached using trypsin, centrifuged, and seeded at a culture density of 3±0.2×10³ cells/cm² in tissue culture flasks, in culture medium lacking gentamicin.
Subsequent Passages: When the culture reached 60-90% confluence, cells were passaged as described above.

### Step 1-3—Cell Concentration, Washing, Formulation, Filling and Cryopreservation

Following the final passage, the resulting cell suspension was centrifuged and re-suspended in culture medium at a final concentration of 20-40×10⁶ cells/milliliter (mL). The cell suspension was diluted 1:1 with 2D Freezing Solution (20% DMSO, 80% FBS), and the cells were cryopreserved in 10% DMSO, 40% FBS, and 50% full DMEM. The temperature was reduced in a controlled rate freezer (1° C./min down to −80° C. followed by 5° C./min down to −120° C.), and the cells were stored in a liquid nitrogen freezer to produce the ICS.

### Production of Cell Product

### Step 2-1: Additional Two-Dimensional (2D) Cell Culturing.

The ICS was thawed, diluted with culture medium, and cultured for up to 10 additional doublings, passaging when reaching 60⁻90% confluence, then were harvested for seeding in the bioreactor.

### Step 2-2: Three Dimensional (3D) Cell Growth in Bioreactor/s

From the cell suspension, 1 or 2 bioreactors were seeded. Each bioreactor contained FibraCel® carriers (New Brunswick Scientific) made of polyester and polypropylene, and culture medium. 170×10⁶ cells were seeded into each 2.8-liter bioreactor.

The growth medium in the bioreactor/s was kept at the following conditions: temp: 37±1° C., Dissolved Oxygen (DO): 70±10% and pH 7.4±0.2. Filtered gases (Air, CO₂, N₂ and O₂) were supplied as determined by the control system in order to maintain the target DO and pH values.

After seeding, the medium was agitated with stepwise increases in the speed, up to 150-200 RPM by 24 hours. Perfusion was initiated several hours after seeding and was adjusted on a daily basis in order to keep the glucose concentration constant at approximately 550 mg\liter.

Cell harvest was performed at the end of the growth phase (approximately day 6). Bioreactors were washed for 1 minute with pre-warmed sterile PBS, and cells were detached. The cells were found to be over 90% maternally-derived cells.

### Step 2-3: Downstream Processing: Cell Concentration, Washing, Formulation, Filling and Cryopreservation

In some experiments, the cell suspension underwent concentration and washing, using suspension solution (5% w/v human serum albumin [HSA] in isotonic solution) as the wash buffer, and diluted 1:1 with 3D-Freezing solution (20% DMSO v/v and 5% HSA w/v in isotonic solution) to a concentration of 10-20×10⁶ cells/ml. In some experiments, a 1:1 mixture of 2D Freezing Solution and full DMEM was used, and the cell concentration was 3-5×10⁶ cells/ml. The temperature of the vials was gradually reduced, and the vials were stored in a gas-phase liquid nitrogen freezer.

### Example 2

### ASC Improve Regeneration of the Hematopoietic System

Methods

On day 0, C57BL/6 mice were sham-irradiated or irradiated with 3 different radiation doses, namely 853, 872, and 904 centigray (cGy), corresponding to LD50, LD70, and LD90, respectively. On days 1 and 5, mice were intramuscularly (IM)-administered 2×10̂6 placental ASC which were predominantly fetal cells. Survival of the mice was recorded.

Results

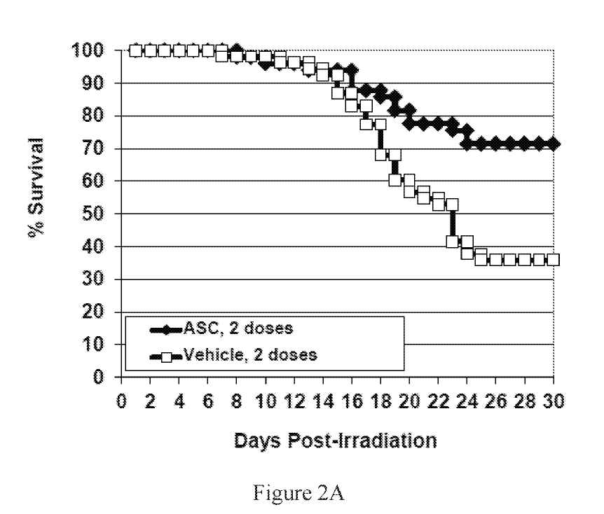

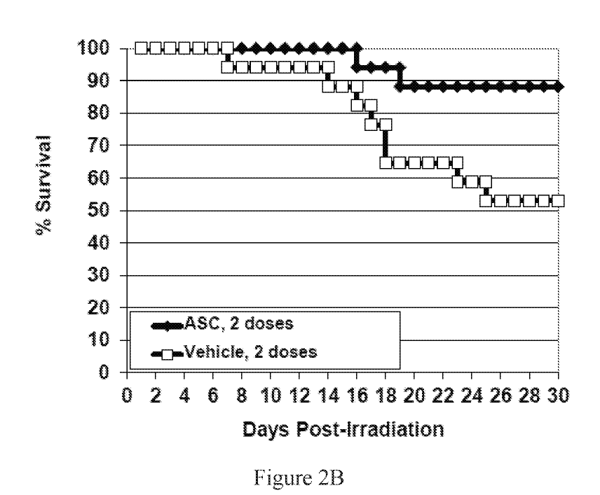

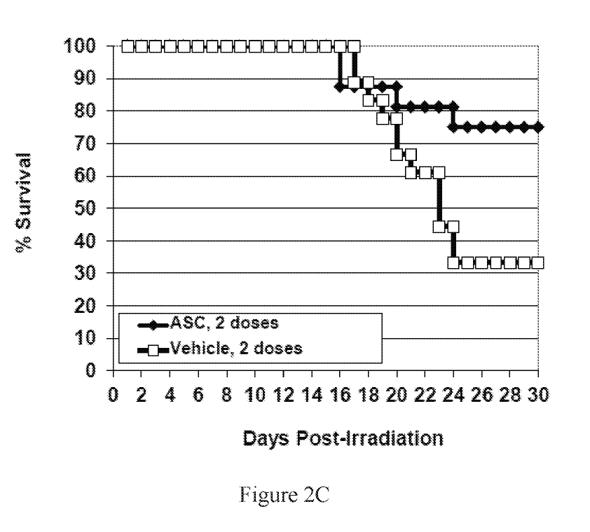

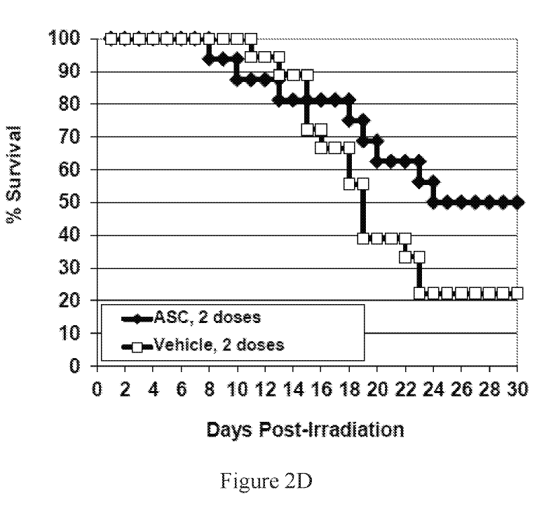

To test the ability of ASC to protect subjects from lethal irradiation, mice were sham-irradiated or irradiated with 3 different radiation doses, namely 853, 872, and 904 cGy, corresponding to LD50, LD70, and LD90, respectively, followed by placental ASC on days 1 and 5. Survival was increased in the ASC-treated mice, as evident when all doses were plotted together (FIG. 2A), or when plotting mice that received the LD50 (FIG. 2B), LD70 (FIG. 2C), and LD90 (FIG. 2D) doses.

### Example 3

### ASC Stimulate Regeneration of Multiple Components of the Hematopoietic System

Methods

Mice were irradiated with an LD70 dose on day zero and were IM-administered 2×10̂6 placental ASC on days 1 and 5. Bone marrow and serum samples were harvested from study mice on days 2, 4, 6, 9, 13 and 23 post-irradiation, and mouse cytokine levels were determined in the samples. Additionally, cells were enumerated and adjusted to reflect the total BM cellularity in the entire mouse. Portions of cells were plated in methylcellulose for determination of HPC content [CFU-GM, burst forming unit-erythroid (BFU-E), and CFU-granulocyte, erythrocyte, monocyte, megakaryocyte (GEMM)]. Frequencies of BM total hematopoietic progenitor cells (HPC) and BM cellularity were used together to calculate the total number of each HPC type in the entire mouse.

Results

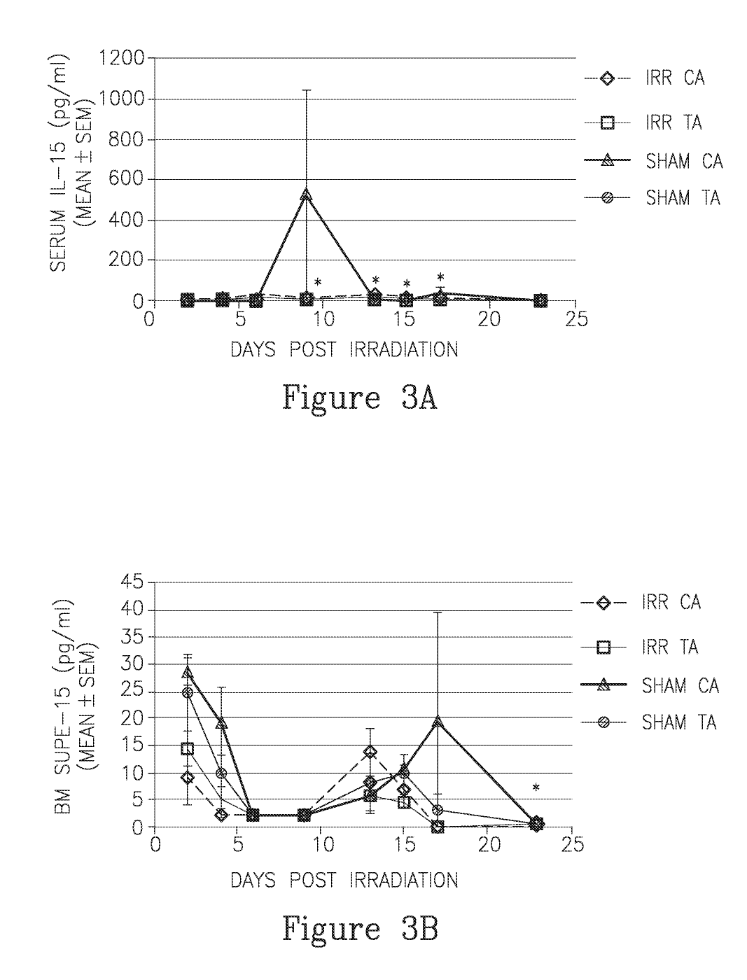

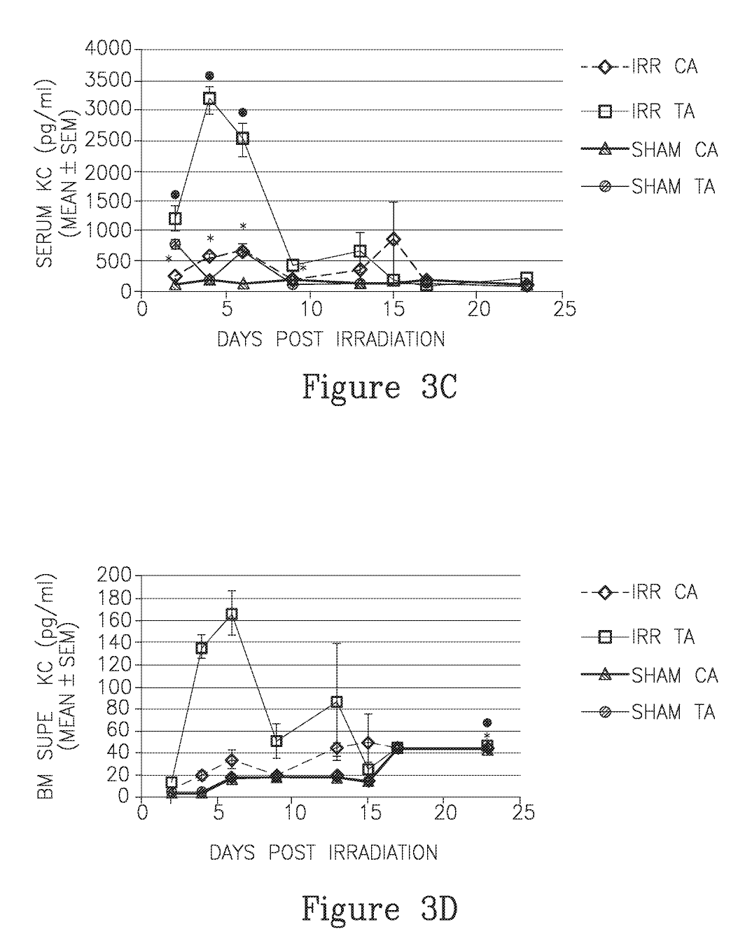

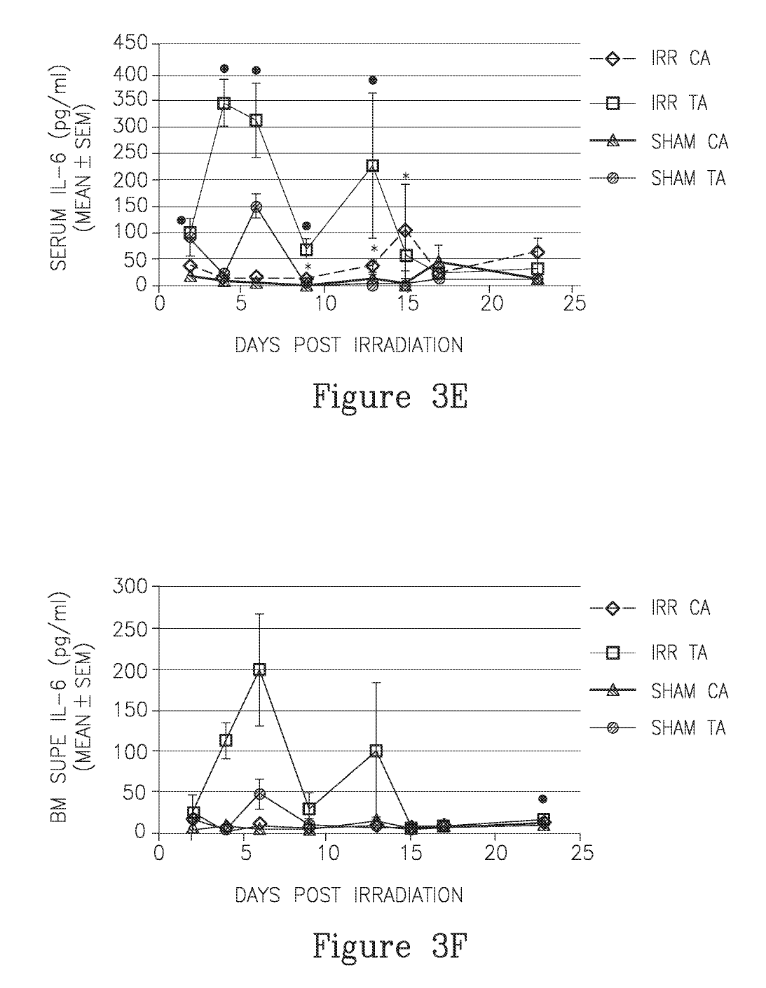

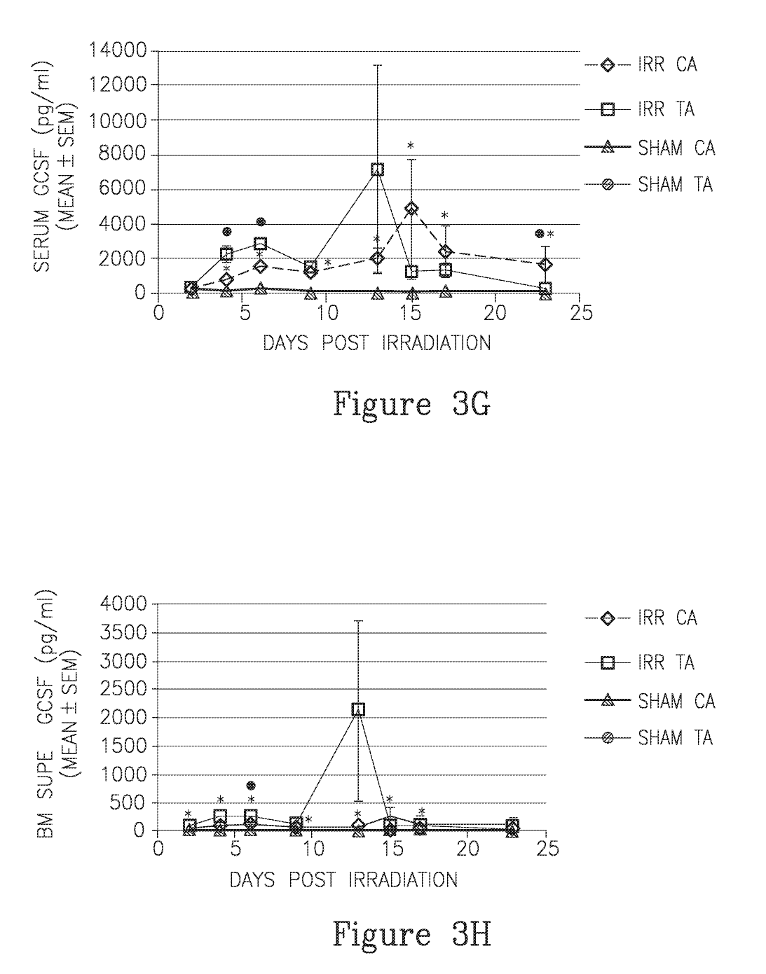

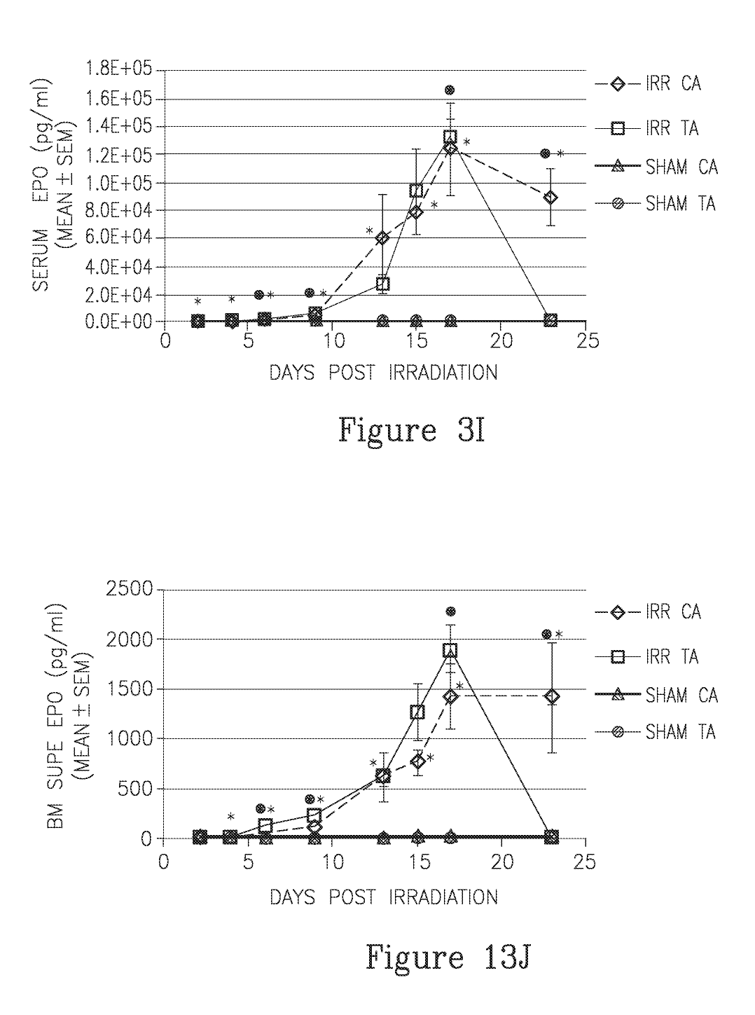

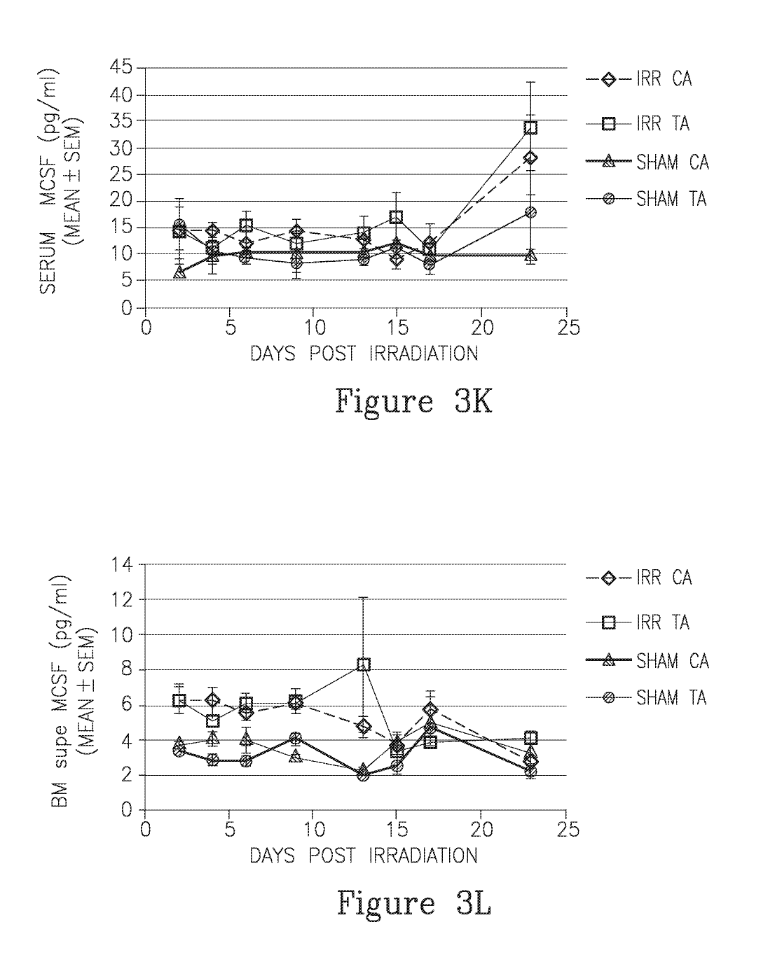

A second study was conducted, having a similar design to that of the previous Example, except that only the LD70 dose was used. Peripheral blood counts, and serum and bone marrow (BM) mouse cytokine levels were measured at various timepoints. The levels of a number of cytokines were followed in the ASC-treated mice (FIGS. 3A-B). The p-values of the differences are shown in Table 1. PLX-R18 treatment significantly increased levels of KC, IL-6, and GM-CSF in the serum and BM of irradiated mice during the first 2 weeks following irradiation.

TABLE 1
p-values of differences in mouse cytokine levels. CA and TA
refer to vehicle- and ASC-treated, respectively. Sham and IRR
refer to sham-irradiated and irradiated mice, respectively.

| | IRR CA vs. IRR TA | | SHAM CA vs. IRR CA | | SHAM CA vs. SHAM TA | |
|---|---|---|---|---|---|---|
| Cytokine | Serum | BM | Serum | BM | Serum | BM |
| IL-15 | *NC | NC | ≦0.0460 | 0.0208 | NC | NC |
| KC | <0.0001 | <0.0001 | ≦0.0108 | 0.0003 | ≦0.0074 | NC |
| IL-6 | ≦0.0328 | <0.0001 | ≦0.0465 | NC | ≦0.0465 | NC |
| G-CSF | ≦0.0202 | 0.0181 | ≦0.0002 | ≦0.0347 | NC | NC |
| EPO | ≦0.0007 | ≦0.0114 | ≦0.0206 | ≦0.0012 | NC | NC |
| M-CSF | NC | NC | NC | 0.0001 | NC | NC |

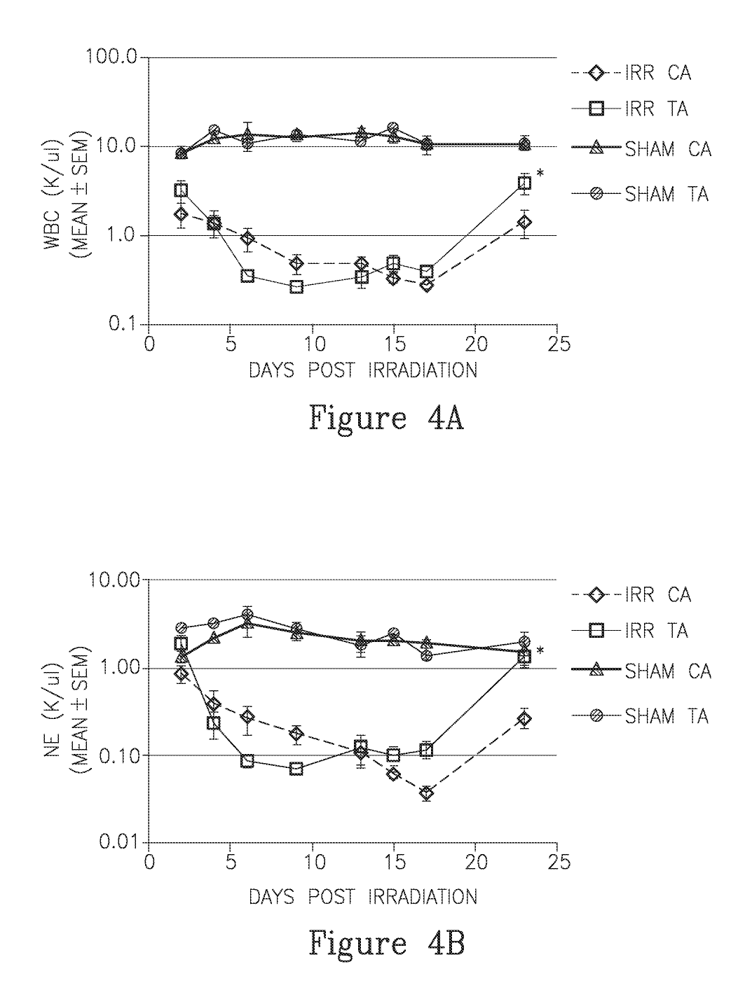

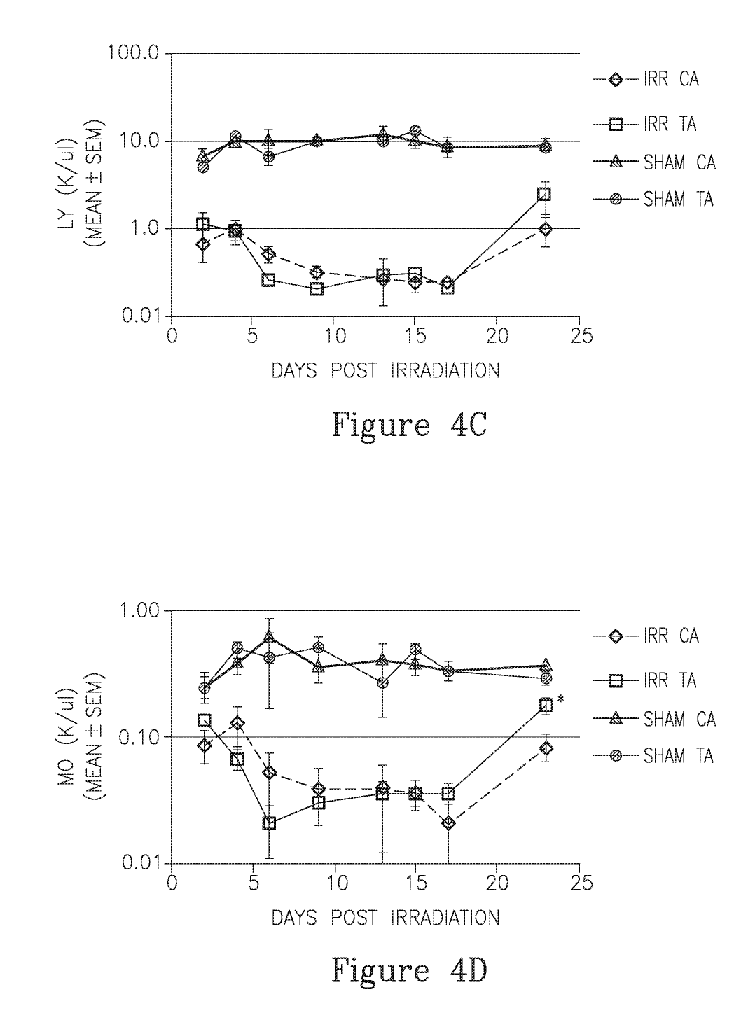

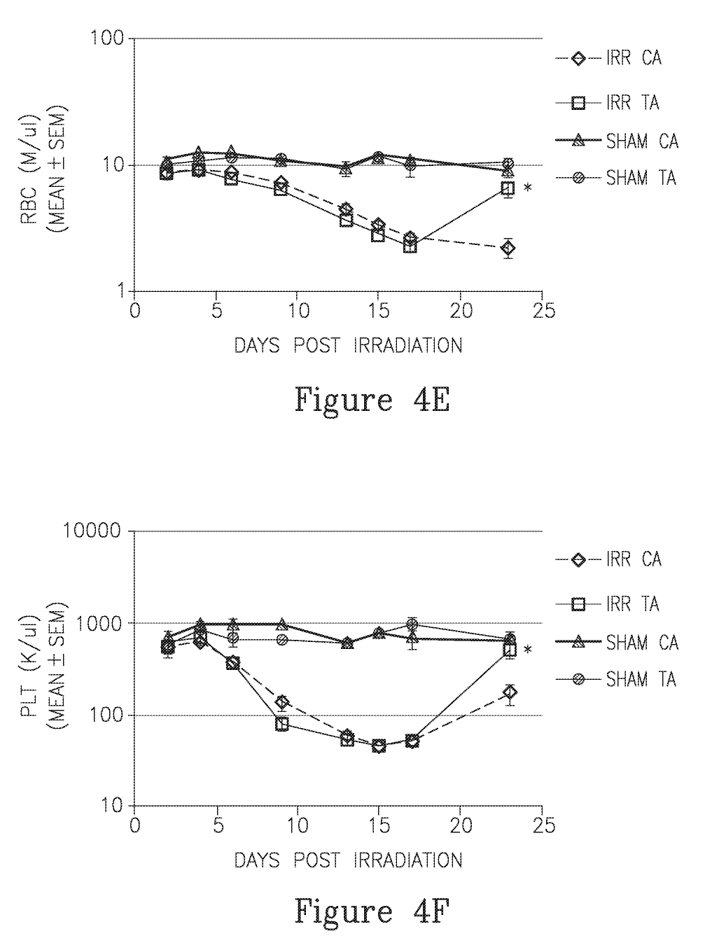

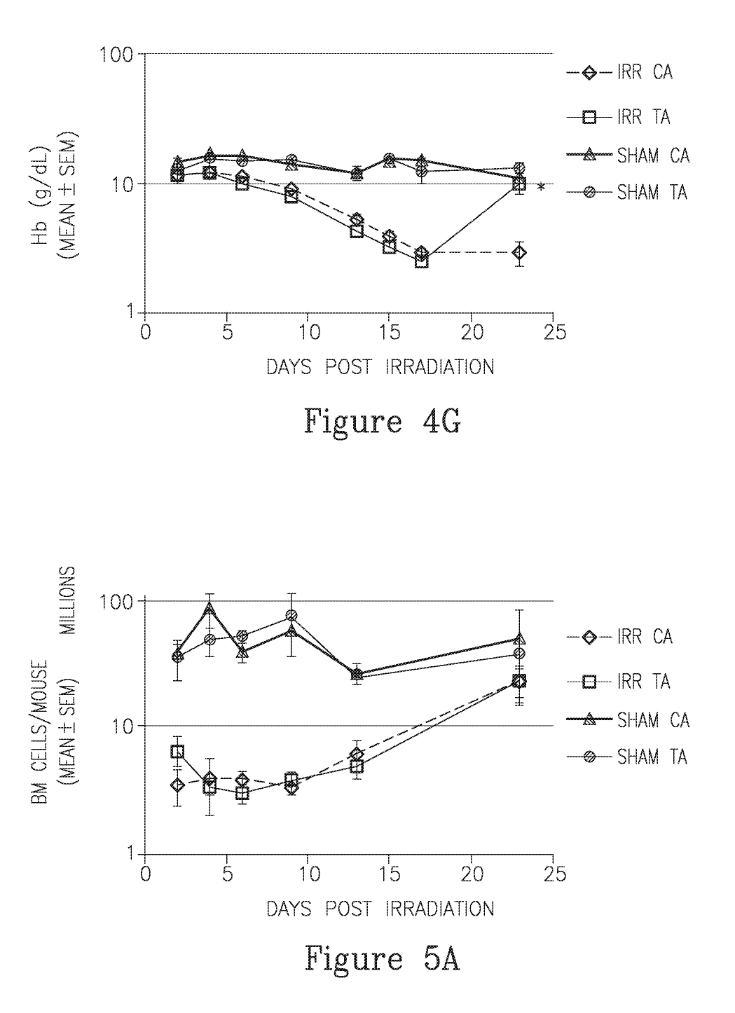

Additionally, FIG. 4 depicts that serum levels of several components were significantly altered by ASC treatment, as shown by plots of white blood cells (A), neutrophils (B), lymphocytes (C), monocytes (D), red blood cells (E), platelets (F), and hemoglobin (G). The serum levels of several components in irradiated mice were increased by PLX-R18 treatment, particularly on the last timepoint at day 23. The p-values of the differences at day 23 are shown in Table 2.

TABLE 2
p-values of differences in blood component levels, between the vehicle-
treated/irradiated and ASC-treated/irradiated groups at day 23.

| WBC | NE | LY | MO | RBC | PLT | Hb | HCT | MCV | % NE |
|---|---|---|---|---|---|---|---|---|---|
| 0.0024 | 0.0026 | 0.1714 | 0.0272 | <0.0001 | 0.0005 | <0.0001 | <0.0001 | <0.0001 | 0.2388 |

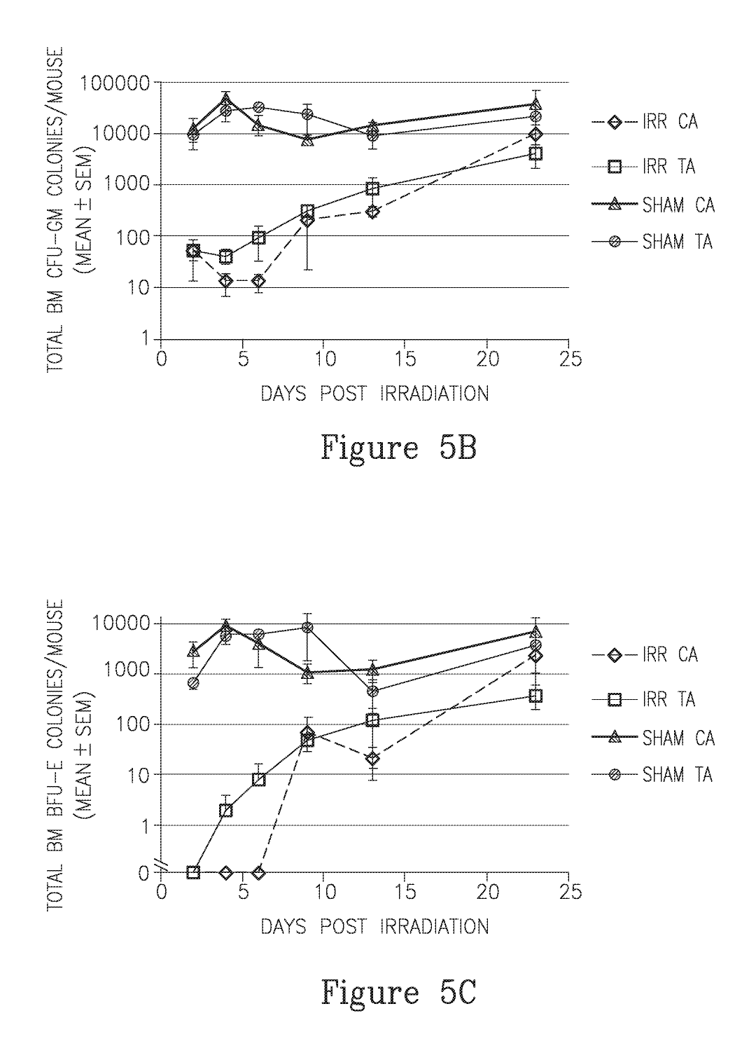

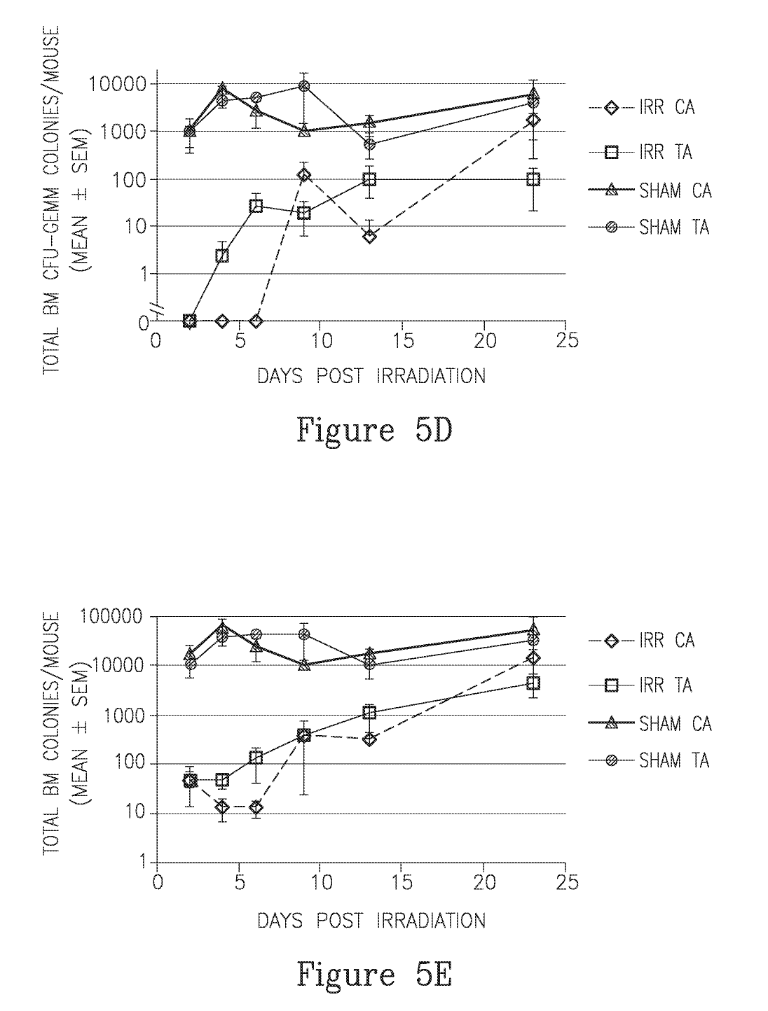

When BM was examined, ASC treatment did not exert a strong effect on BM cellularity (FIG. 5A). FIG. 5 further shows that frequencies of several types of precursors were altered by ASC treatment, as shown by plots of CFU-GM (B), BFU-E (C), CFU-GEMM (D), and BM total HPC (E).

### Example 4

### ASC Enhance Engraftment of Syngeneic and Haploidentical Hematopoietic Transplants

Methods

C57BL/6 mice were lethally irradiated (1000 cGy) and then reconstituted with 4×10⁶ or 8×10⁶ C57BL/6 (syngeneic) BM cells 20 hours (hr) after radiation. 20 hr and 8 days post-radiation, mice were IM-administered 10̂6 placental ASC which were predominantly fetal cells. In another study, F1 (BALB/c×C57BL/6) mice were lethally irradiated and then reconstituted with 2×10⁶ or 4×10⁶ C57BL/6 (haploidentical) BM cells 20 hr after radiation. 20 hr and 8 days post-radiation, mice were IM-administered 10̂6 placental ASC. Weight, survival, and blood and marrow components were assessed at various timepoints.

Results

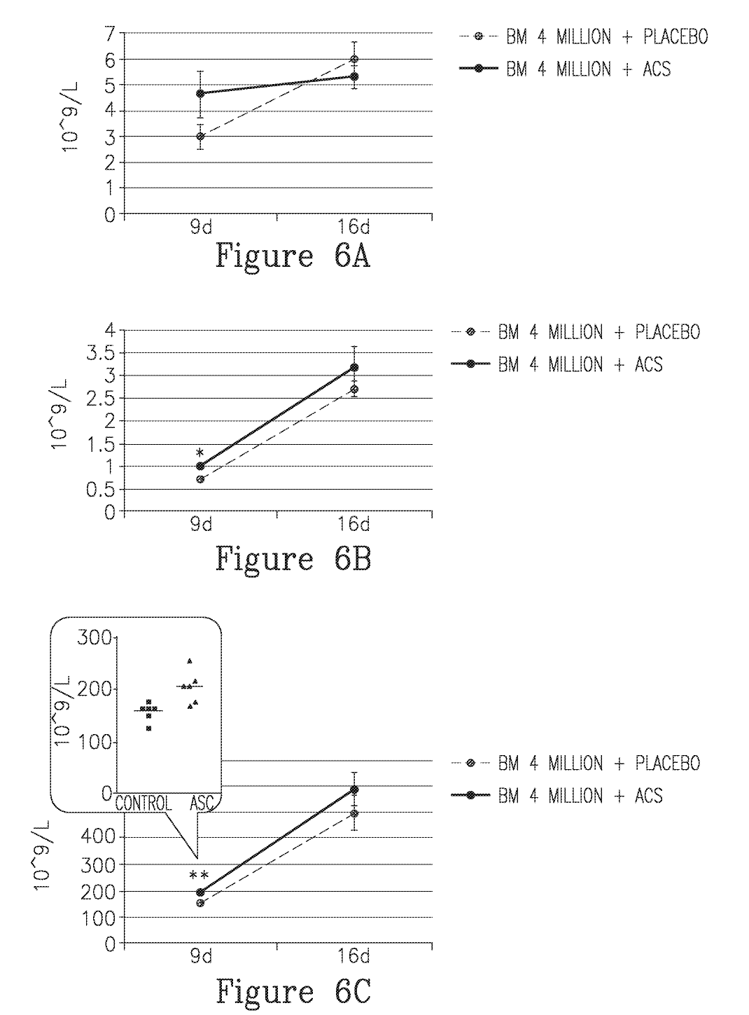

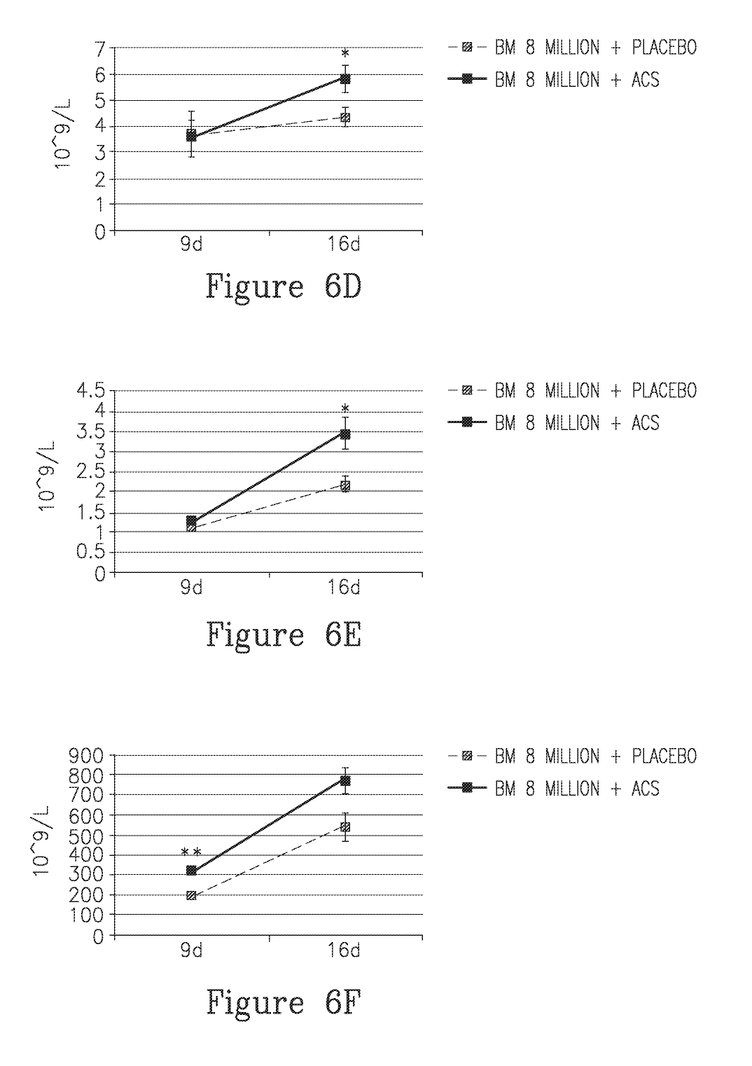

In the syngeneic study, various blood components were altered in the ASC-treated mice, as shown in FIG. 6, which contains plots of white blood cells (A), granulocytes (B), and platelets (C) in the low-dose group, as well as plots of white blood cells (D), granulocytes (E), and platelets (F) in the high-dose group.

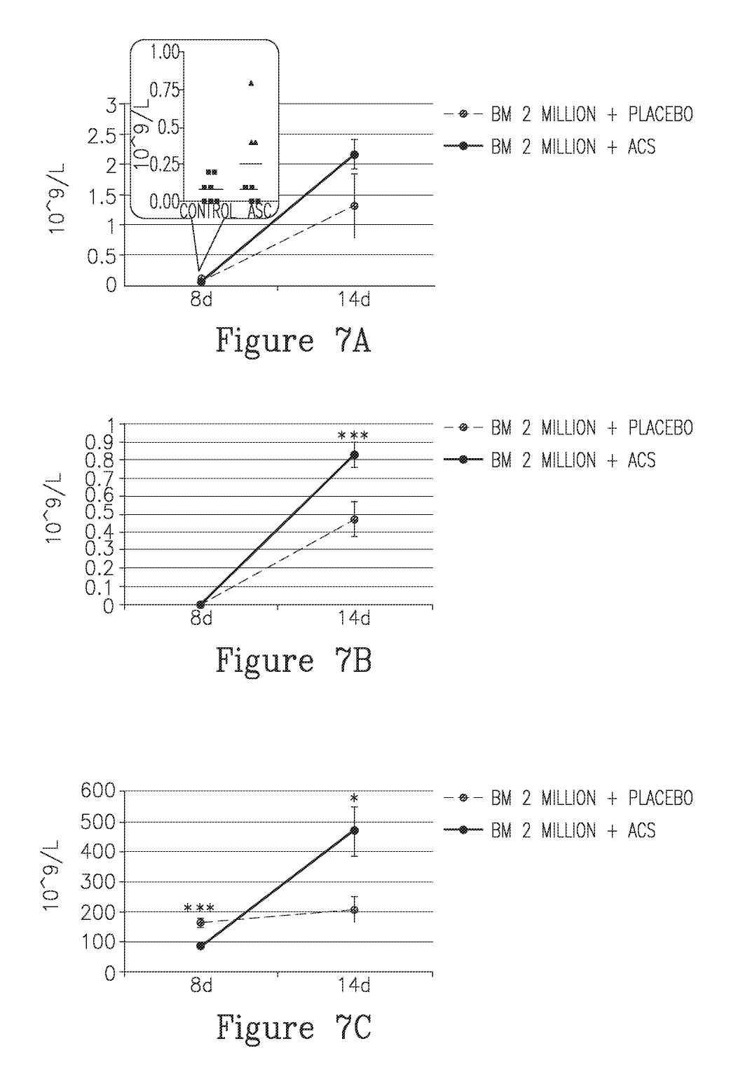

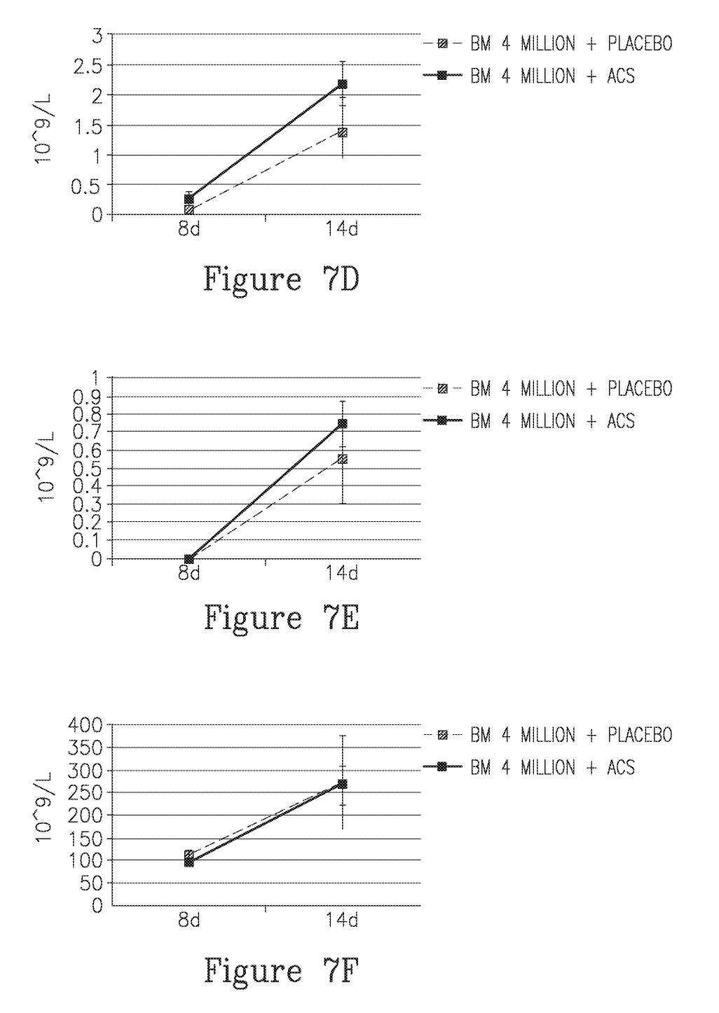

Similar trends were observed in the haploidentical study, as shown in FIG. 7, which contains plots of white blood cells (A), granulocytes (B), and platelets (C) in the low-dose group, as well as plots of white blood cells (D), granulocytes (E), and platelets (F) in the high-dose group.

### Example 5

### Enhancement of Xenogeneic Transplant Engraftment by ASC

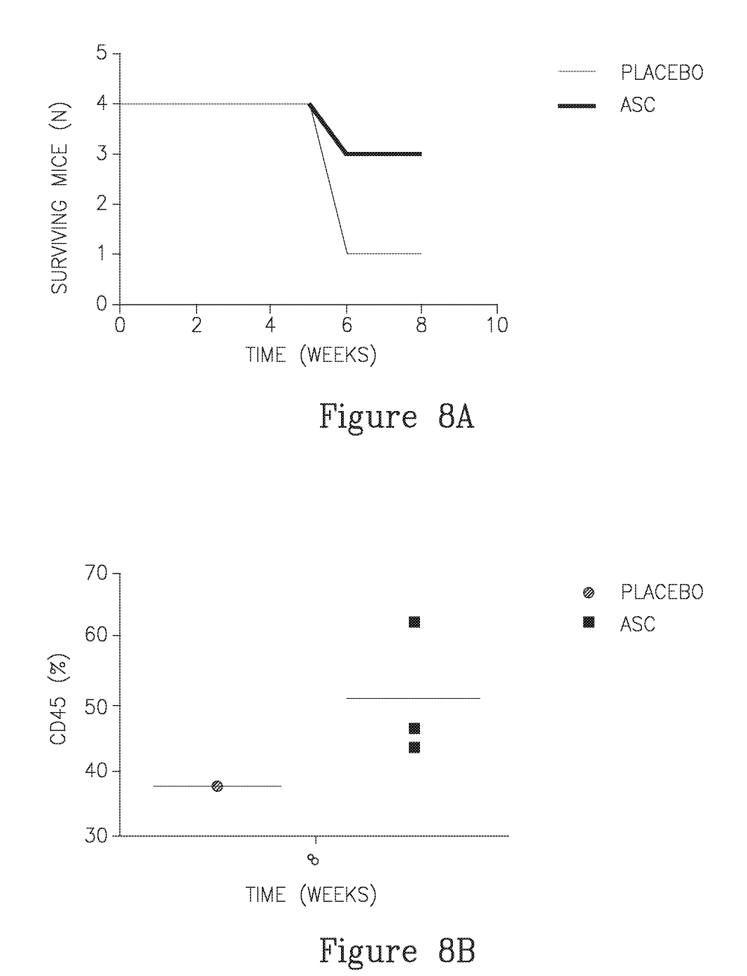

C57BL/6 mice were non-lethally irradiated (300 cGy) and then reconstituted with 5×10⁵ human (xenogeneic) BM cells 20 hours (hr) after radiation. 2 and 7 days post-radiation, mice were IM—or IV-administered 10̂6 placental ASC which were predominantly fetal cells. Survival and the extent of engraftment were assessed at various timepoints. The mice that received ASC had an improved survival curve (FIG. 8A) and exhibited more human HSC in their BM 8 weeks after irradiation (FIG. 8B).

### Example 6

### Conditioned Medium from ASC Induces Migration of BM Cells

Methods

2 populations of placental ASC were utilized, one primarily maternal (population #1), and the other predominantly fetal (population #2). The ASC were thawed, re-suspended in DMEM supplemented with 10% FBS and 2 mM L-Glutamine, and cultured for 24 hr in a humidified incubator (5% CO₂, 95% air at 37° C.). After 24 hr, the medium was replaced with RPMI 1640, with L-Glutamine (Ref 01-100-1, Biological Industries) supplemented with 0.5% HAS, and the cells were cultured for additional 24 hr. Then the conditioned medium (CM) was collected from the plate and centrifuged at 4566 g, 4° C. for 1 min to remove cell debris.

Murine BM cells were seeded on the upper insert of a 24 well-Transwell® plate, with a membrane having 5-micron pores. 0.5 ml of CM or RPMI medium (which served as a negative control) were added to the lower chamber of the Transwell® plate. The cells were incubated in a humidified incubator (5% CO2, 95% air at 37° C.) for 24 hr, and then upper inserts were gently removed, and the migrated cells were collected from the lower chambers and counted by CyQuant® NF, a fluorescent, DNA-specific dye.

Results

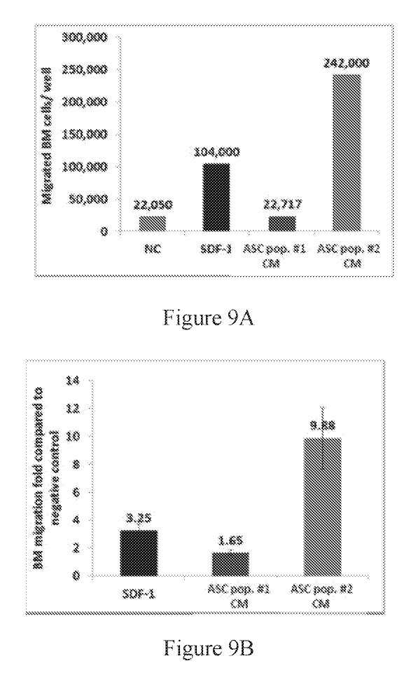

ASC CM induced a nearly 10-fold higher migration rate of BM cells through a 5μ Transwell® insert towards the CM, compared to the negative control (unaltered medium; FIG. 9A). Moreover, this migration rate was about 3-fold higher that the migration rate towards positive control medium, supplemented with 100 ng/ml SDF-1 (FIG. 9B).

### Example 7

### ASC Reduce the LD50 of Acute Radiation

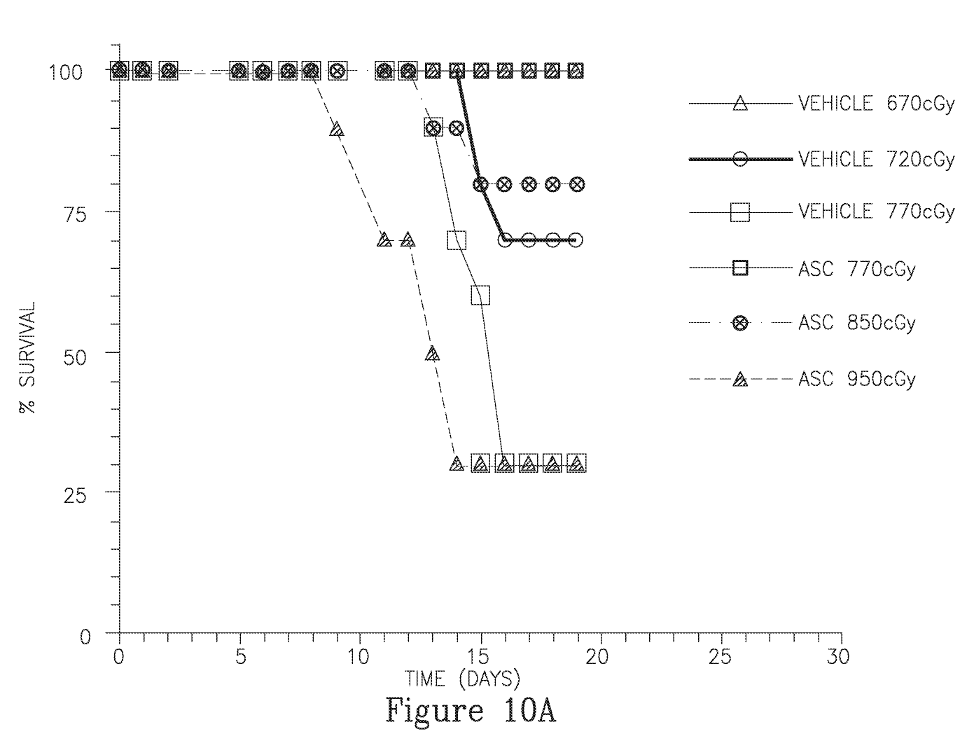

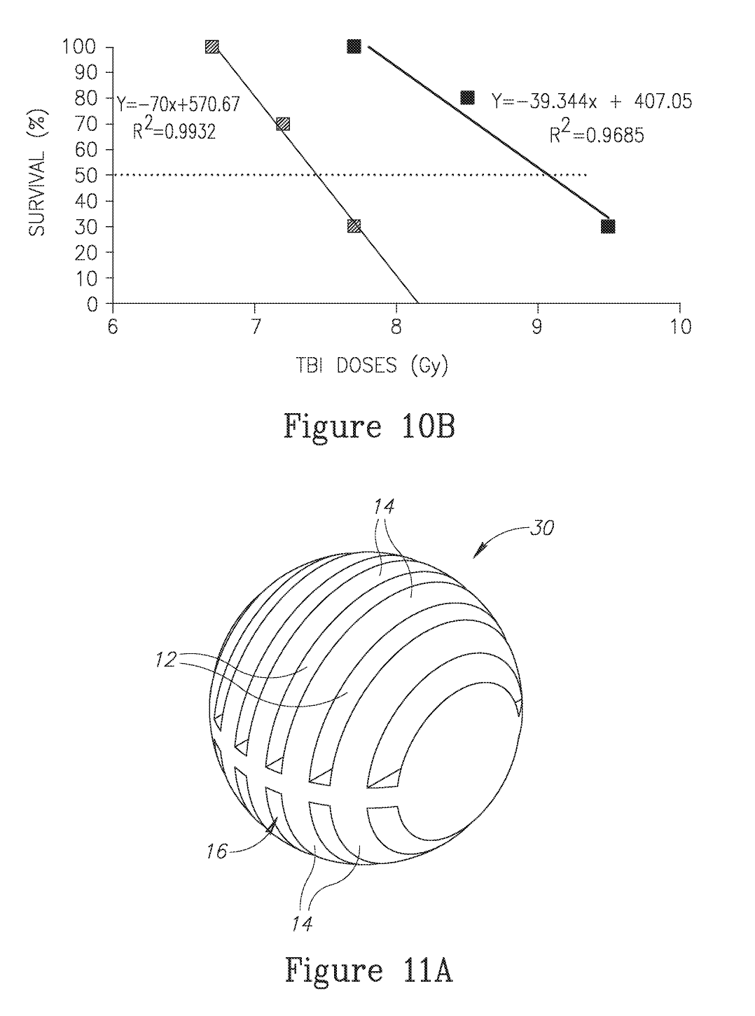

C3H mice were exposed to total body radiation at a dose of 670 cGy (n=10; vehicle), 720 cGy (n=10; vehicle), 770 cGy (n=20; 10 vehicle and 10 ASC), 850 cGy (n=10; ASC), or 950 cGy (n=10; ASC). 24 hours and 5 days after the irradiation, the mice indicated above as receiving ASC were injected IM with 2×10⁶ ASC cells in 100 microliters (mcl) plasmaLyte A/mouse. The remaining 30 mice were injected with the same volume of plasmaLyte A (vehicle). As in previous studies, the treated dose exhibited reduced mortality per dose (FIG. 10A). The LD₅₀ for the treated mice was 907.5 cGy, while the LD₅₀ for the untreated mice was 907.5 cGy 743.8, yielding a dose reduction factor of 1.22 (FIG. 10B).

### Example 8

### Use of ASC in Treating Incomplete Engraftment

Subjects with delayed or incomplete engraftment, as defined in Trébéden-Negre H et al, are administered ASC, typically between 1-24 months after the transplant. In other experiments, the ASC may be administered together with an additional transplant. Amelioration of the disorder is evidence of therapeutic efficacy.

### Example 9

### Use of ASC in Enhancing Hematopoiesis Following an RIC HSC Transplant

ASC are tested in an animal model of hematopoiesis following a reduced intensity conditioning (RIC) HSC transplant, for example as described in Chandrasekaran D et al, Koyama M et al, and the references cited therein. In still other experiments, human subjects having received an RIC HSC are administered the described cells, for example as a single infusion within 14 days of receiving the transplant, or as 2-5 separate infusions over a 1-4 month period, within 3 months of the transplant. Amelioration of the disorder is evidence of therapeutic efficacy.

### Example 10

### Use of ASC in Treating MDS

ASC are tested in an animal model of myelodysplastic syndrome (MDS), for example as described in Inoue D et al, Li X et al, and the references cited therein. In other experiments, human subjects with MDS are administered the described cells. Amelioration of the disorder is evidence of therapeutic efficacy. In still other experiments, the effect of ASC on the incidence of acute myeloid leukemia (AML) is assessed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References may be Cited in Text

Bacigalupo A. Third EBMT/AMGEN Workshop on reduced-intensity conditioning allogeneic haemopoietic stem cell transplants (RIC-HSCT), and panel consensus. Bone Marrow Transplant. 33(7),691-696 (2004).

Chandrasekaran D et al, Modeling promising nonmyeloablative conditioning regimens in nonhuman primates. Hum Gene Ther. 2014;25(12):1013-22.

Clayton A et al, Analysis of antigen presenting cell derived exosomes, based on immuno-magnetic isolation and flow cytometry. J Immunol Methods. 2001;247(1-2):163-74.

Crescitelli R et al, Distinct RNA profiles in subpopulations of extracellular vesicles: apoptotic bodies, microvesicles and exosomes. J Extracell Vesicles. 2013 Sep 12;2.

Fenaux Pet al, Cytogenetics of myelodysplastic syndromes. Semin Hematol 33(2):127-138, 1996.

Horwitz M E et al, Umbilical cord blood expansion with nicotinamide provides long-term multilineage engraftment. J Clin Invest. 2014 July;124(7):3121-8.

Inoue D et al, Myelodysplastic syndromes are induced by histone methylation-altering ASXL1 mutations. J Clin Invest. 2013 November;123(11):4627-40.

Koyama M, Expansion of donor-reactive host T cells in primary graft failure after allogeneic hematopoietic SCT following reduced-intensity conditioning. Bone Marrow Transplant. 2014;49(1):110-5.

Li X et al, Murine xenogeneic models of myelodysplastic syndrome: an essential role for stroma cells. Exp Hematol. 2014 January;42(1):4-10.

Mathias R A et al, Isolation of extracellular membranous vesicles for proteomic analysis. Methods Mol Biol. 2009;528:227-42.

Paquette R L et al: N-ras mutations are associated with poor prognosis and increased risk of leukemia in myelodysplastic syndrome. Blood 82(2):590-599, 1993.

Paquette R L. Diagnosis and management of aplastic anemia and myelodysplastic syndrome. Oncology (Williston Park). 2002 September;16(9 Suppl 10):153-61.

Trébéden-Negre H et al, Delayed recovery after autologous peripheral hematopoietic cell transplantation: potential effect of a high number of total nucleated cells in the graft. Transfusion. 2010 December;50(12):2649-59.

Weisdorf D J et al, Hematopoietic growth factors for graft failure after bone marrow transplantation: a randomized trial of granulocyte-macrophage colony-stimulating factor (GM-CSF) versus sequential GM-CSF plus granulocyte-CSF. Blood. 1995 June 15;85(12):3452-6

1. A method of treating incomplete engraftment of a hematopoietic stem cell (HSC) transplant in a subject in need thereof, comprising the step of administering to said subject a pharmaceutical composition comprising adherent stromal cells (ASC), wherein said ASC are derived from a placenta or from adipose tissue, thereby treating incomplete engraftment.

2. The method of claim 1, wherein said HSC are derived from blood or bone marrow.

3. The method of claim 1, wherein said HSC transplant is a cord blood transplant.

4. The method of claim 1, wherein said HSC transplant is an autologous transplant.

5. The method of claim 1, wherein said HSC transplant is an allogenic transplant.

6. A method of enhancing hematopoiesis in a subject having received a reduced intensity conditioning (RIC) HSC transplant, comprising the step of administering to said subject a pharmaceutical composition comprising adherent stromal cells (ASC), wherein said ASC are derived from a placenta or from adipose tissue, thereby enhancing hematopoiesis in a subject having received an RIC transplant.

7-10. (canceled)

11. The method of claim 6, wherein said HSC transplant is an allogenic transplant.

12. A method of treating myelodysplastic syndrome (MDS) in a subject in need thereof, comprising the step of administering to said subject a pharmaceutical composition comprising adherent stromal cells (ASC), wherein said ASC are derived from a placenta or from adipose tissue, thereby treating MDS.

13-25. (canceled)

26. The method of claim 1, wherein said ASC have been incubated in a 3D culture apparatus.

27. The method of claim 26, further comprising the subsequent step of harvesting said ASC by removing said ASC from said 3D culture apparatus.

28. The method of claim 27, wherein said ASC have been incubated in a 2D adherent-cell culture apparatus, prior to said incubation in a 3D culture apparatus.

29. The method of claim 1, wherein said 3D culture apparatus comprises a bioreactor.

30. The method of claim 1, wherein said 3D culture apparatus comprises a synthetic adherent material.

31. The method of claim 30, wherein said synthetic adherent material is a fibrous matrix.

32. The method of claim 30, wherein said synthetic adherent material is selected from the group consisting of a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber.

33. The method of claim 1, wherein said 3D culture apparatus comprises microcarriers.

34-42. (canceled)

43. The method of claim 1, wherein said ASC originate from placenta tissue.

44. The method of claim 1, wherein said ASC originate from adipose tissue.

45. The method of claim 1, wherein said ASC express a marker selected from the group consisting of CD73, CD90, CD29 and CD105.

46. The method of claim 1, wherein said ASC do not express a marker selected from the group consisting of CD3, CD4, CD80, CD11b, CD14, CD19, and CD34.
